(19) United States
(12) Patent Application Publication
(10) Pub. No.: US 2023/0282314 A1
(43) Pub. Date: Sep. 7, 2023

(54) CHARACTERIZING FUNCTIONAL REGULATORY ELEMENTS USING MACHINE LEARNING

(71) Applicant: Camp4 Therapeutics Corporation, Cambridge, MA (US)

(72) Inventor: Yuchun Guo, Framingham, MA (US)

(21) Appl. No.: 18/177,284

(22) Filed: Mar. 2, 2023

Related U.S. Application Data
(60) Provisional application No. 63/380,837, filed on Oct. 25, 2022.
(60) Provisional application No. 63/315,962, filed on Mar. 2, 2022.

(51) Int. Cl. G16B 40/20

(52) U.S. Cl. CPC G16B 40/20

(57) ABSTRACT

Disclosed herein are methods for implementing machine learning models to analyze features from epigenomic datasets to determine whether enhancer-promoter pairs are functional or non-functional. Features can include a first set of features extracted from the epigenomic datasets. Furthermore, features can include a second set of features engineered from features of the first set. Machine learning models that incorporate features, including the first set of features and engineered second set of features, can predict, with improved metrics, whether enhancer-promoter pairs are functional or non-functional.

### CROSS-REFERENCE TO RELATED APPLICATIONS

This application claims the benefit of and priority to U.S. Provisional Pat. Application No. 63/315,962 filed Mar. 2, 2022 and U.S. Provisional Pat. Application No. 63/380,837 filed Oct. 25, 2022, the entire disclosure of each of which is hereby incorporated by reference in its entirety for all purposes.

### BACKGROUND

Transcriptional enhancers control how genes are expressed in specific cell types. Enhancer disruption and misregulation are implicated as disease-driving mechanisms. Modalities that specifically target enhancers that control disease-associated genes are being pursued to develop new drugs for a range of indications. However, it remains a major challenge to link functional enhancers to their target genes. Conventional methods for determining functional enhancer-promoter (E-P) pairs such as the Activity by Contact (ABC) model, as described in Fulco et al. (2019) Nat. Genet. 51: 1664-9, attempt to predict functional E-P interactions with limited information, resulting in varying success. There is a need for methods that predict functional E-P interactions with improved accuracy.

### SUMMARY OF THE INVENTION

Disclosed herein are methods for implementing machine learning models to analyze various features from epigenomic datasets to determine whether enhancer-promoter (E-P) pairs are functional or non-functional. In various embodiments, different types of features, including specifically engineered features, are incorporated in machine learning models. In particular, features of the machine learning model include a first set of features extracted from the epigenomic datasets and further include a second set of features engineered from features of the first set. Thus, the features disclosed herein enable machine learning models to more accurately predict functional E-P pairs in comparison to conventional methods (e.g., the ABC model).

Disclosed herein is a method, comprising: obtaining a dataset comprising epigenomic data for one or more enhancer-promoter pairs; for the one or more enhancer-promoter pairs: generating, from the dataset comprising epigenomic data, values for a plurality of features comprising a first set of features and a second set of features of the enhancer-promoter pair by: generating values for the first set of features; and generating values for the second set of features engineered from subsets of the first set of features; applying a machine learning model to analyze the values for the plurality of features of the one or more enhancer-promoter pairs; and determining whether one of the one or more enhancer-promoter pairs is a functional enhancer-promoter pair based on an output of the machine learning model.

In various embodiments, the second set of features engineered from subsets of the first set of features comprise an enhancer contribution feature that quantifies relative contribution of the enhancer across a plurality of enhancers to a gene operably controlled by the promoter. In various embodiments, the second set of features further comprise a composite feature of the enhancer representing a combination of an ATAC feature, an EP300 feature, a H3K4me1 feature, and a HiChIP feature. In various embodiments, the enhancer contribution feature is a ratio of the composite feature of the enhancer to a combination of a plurality of composite features for the enhancer. In various embodiments, the second set of features engineered from subsets of the first set of features comprise a gene contribution feature that quantifies relative contribution of a gene operably controlled by the promoter across a plurality of genes influenced by the enhancer. In various embodiments, the second set of features further comprise a composite feature of the gene representing a combination of an ATAC feature, an EP300 feature, a H3K4me1 feature, and a HiChIP feature. In various embodiments, the gene contribution feature is a ratio of the composite feature of the gene to a combination of a plurality of composite features for the gene.

In various embodiments, the second set of features comprise at least three features. In various embodiments, the second set of features comprise APMI, fracEnh, and fracGene features. In various embodiments, the second set of features comprise nine features. In various embodiments, the second set of features comprise APMI, fracEnh, fracGene, fracGmE, fracGpE, apmiGene, apmiEnh, apmiGmE, and apmiGpE features. In various embodiments, the first set of features comprise 35 or more features. In various embodiments, the first set of features comprise features of ATAC, EP300, H3K4me1, HiChIP, and genomic distance. In various embodiments, the first set of features comprise 75 or more features.

In various embodiments, at least one feature of the second set has a higher feature importance value in comparison to at least one feature of the first set. In various embodiments, at least three features of the second set have a higher feature importance value in comparison to at least three features of the first set. In various embodiments, at least five features of the second set have a higher feature importance value in comparison to at least five features of the first set. In various embodiments, each feature of the second set has a higher feature importance value in comparison to each feature of the first set.

In various embodiments, the machine learning model achieves an area under a precision recall curve (AUPR) metric of at least 0.55. In various embodiments, the machine learning model achieves an area under a precision recall curve (AUPR) metric of at least 0.60. In various embodiments, the machine learning model achieves an area under a receiver operative curve (AUROC) metric of at least 0.90. In various embodiments, the machine learning model achieves an area under a receiver operative curve (AUROC) metric of at least 0.91. In various embodiments, the machine learning model is a random forest model.

In various embodiments, the dataset comprises one or more of: chromatin accessibility data identifying chromatin-accessible regions across the genome; and chromatin binding data identifying chromatin interactions. In various embodiments, the chromatin accessibility data comprises DNase-seq or ATAC-seq data. In various embodiments, the chromatin binding data comprises data for one or more of: DNA-DNA interactions; chromatin domains; protein-chromatin binding sites; and transcription factor binding motifs.

In various embodiments, the chromatin binding data comprising HiChIP or ChIP-seq data. In various embodiments, the chromatin binding data comprises data for one or more active enhancer marks. In various embodiments, the one or more active enhancer marks comprise EP300, H3K27ac or H3K4me1.

In various embodiments, the chromatin binding data comprises data for one or more repressive factors. In various embodiments, the one or more repressive factors comprise H3K27me3, H3K9me3, H4K20me1, NCOR1, HDAC1/2/3, EZH2, SUZ12, ZEB2, or REST.

In various embodiments, the machine learning model is trained using training data derived from a first cell type, and wherein the dataset comprising epigenomic data is derived from a second cell type different from the first cell type. In various embodiments, the training data are generated by performing an enhancer-based perturbation screen to cells of the first cell type. In various embodiments, the enhancer-based perturbation screen is a CRISPRi-based or CRISPRa-based enhancer perturbation screen.

Additionally disclosed herein is a non-transitory computer readable medium, comprising instructions that, when executed by a processor, cause the processor to: obtain a dataset comprising epigenomic data for one or more enhancer-promoter pairs; for the one or more enhancer-promoter pairs: generate, from the dataset comprising epigenomic data, values for a plurality of features comprising a first set of features and a second set of features of the enhancer-promoter pair by: generating values for the first set of features; and generating values for the second set of features engineered from subsets of the first set of features; apply a machine learning model to analyze the values for the plurality of features of the one or more enhancer-promoter pairs; and determine whether one of the one or more enhancer-promoter pairs is a functional enhancer-promoter pair based on an output of the machine learning model.

In various embodiments, the second set of features engineered from subsets of the first set of features comprise an enhancer contribution feature that quantifies relative contribution of the enhancer across a plurality of enhancers to a gene operably controlled by the promoter. In various embodiments, the second set of features further comprise a composite feature of the enhancer representing a combination of an ATAC feature, an EP300 feature, a H3K4me1 feature, and a HiChIP feature. In various embodiments, the enhancer contribution feature is a ratio of the composite feature of the enhancer to a combination of a plurality of composite features for the enhancer. In various embodiments, the second set of features engineered from subsets of the first set of features comprise a gene contribution feature that quantifies relative contribution of a gene operably controlled by the promoter across a plurality of genes influenced by the enhancer. In various embodiments, the second set of features further comprise a composite feature of the gene representing a combination of an ATAC feature, an EP300 feature, a H3K4me1 feature, and a HiChIP feature. In various embodiments, the gene contribution feature is a ratio of the composite feature of the gene to a combination of a plurality of composite features for the gene.

In various embodiments, the second set of features comprise at least three features. In various embodiments, the second set of features comprise APMI, fracEnh, and fracGene features. In various embodiments, the second set of features comprise nine features. In various embodiments, the second set of features comprise APMI, fracEnh, fracGene, fracGmE, fracGpE, apmiGene, apmiEnh, apmiGmE, and apmiGpE features. In various embodiments, the first set of features comprise 35 or more features. In various embodiments, the first set of features comprise features of ATAC, EP300, H3K4me1, HiChIP, and genomic distance. In various embodiments, the first set of features comprise 75 or more features.

In various embodiments, at least one feature of the second set has a higher feature importance value in comparison to at least one feature of the first set. In various embodiments, at least three features of the second set have a higher feature importance value in comparison to at least three features of the first set. In various embodiments, at least five features of the second set have a higher feature importance value in comparison to at least five features of the first set. In various embodiments, each feature of the second set has a higher feature importance value in comparison to each feature of the first set.

In various embodiments, the machine learning model achieves an area under a precision recall curve (AUPR) metric of at least 0.55. In various embodiments, the machine learning model achieves an area under a precision recall curve (AUPR) metric of at least 0.60. In various embodiments, the machine learning model achieves an area under a receiver operative curve (AUROC) metric of at least 0.90. In various embodiments, the machine learning model achieves an area under a receiver operative curve (AUROC) metric of at least 0.91.

In various embodiments, the machine learning model is a random forest model. In various embodiments, the dataset comprises one or more of: chromatin accessibility data identifying chromatin-accessible regions across the genome; and chromatin binding data identifying chromatin interactions. In various embodiments, the chromatin accessibility data comprises DNase-seq or ATAC-seq data. In various embodiments, the chromatin binding data comprises data for one or more of: DNA-DNA interactions; chromatin domains; protein-chromatin binding sites; and transcription factor binding motifs. In various embodiments, the chromatin binding data comprising HiChIP or ChIP-seq data. In various embodiments, the chromatin binding data comprises data for one or more active enhancer marks. In various embodiments, the one or more active enhancer marks comprise EP300, H3K27ac or H3K4me1.

In various embodiments, the chromatin binding data comprises data for one or more repressive factors. In various embodiments, the one or more repressive factors comprise H3K27me3, H3K9me3, H4K20me1, NCOR1, HDAC1/2/3, EZH2, SUZ12, ZEB2, or REST.

In various embodiments, the machine learning model is trained using training data derived from a first cell type, and wherein the dataset comprising epigenomic data is derived from a second cell type different from the first cell type. In various embodiments,

In various embodiments, the training data are generated by performing an enhancer-based perturbation screen to cells of the first cell type. In various embodiments, the enhancer-based perturbation screen is a CRISPRi-based or CRISPRa-based enhancer perturbation screen.

### BRIEF DESCRIPTION OF THE SEVERAL VIEWS OF THE DRAWINGS

These and other features, aspects, and advantages of the present invention will become better understood with regard to the following description, and accompanying drawings, where:

Figure FIG. 1 depicts a block diagram of an example regulatory element characterization system, in accordance with an embodiment.

FIG. 2A depicts example data (e.g., EP300, ATAC-seq, H3K27ac, K3K4me1) of an epigenomic dataset, in accordance with an embodiment.

FIG. 2B depicts a flow diagram for characterizing regulatory elements, in accordance with an embodiment.

FIG. 3A is a flow process for characterizing regulatory elements, in accordance with an embodiment.

FIG. 3B shows example determination of values of engineered features, in accordance with an embodiment.

FIG. 4 illustrates an example computer for implementing the entities shown in FIG. 1.

FIG. 5 is a diagram showing example training and deployment of a machine learning model for inferring functional enhancer-promoter pairs.

FIG. 6A is an example diagram showing CRISPRi screening for generating training datasets.

FIG. 6B shows example generation and implementation of a machine learning model to predict enhancer-promoter and enhancer-gene interactions.

FIG. 7A shows differential features that distinguish functional and non-functional enhancer-promoter pairs.

FIG. 7B shows ranking of features according to feature importance.

FIG. 8A is a precision-recall curve of the “EPIC full” and “EPIC basic” models in comparison to a state of the art “ABC” model.

FIG. 8B shows performance of the “EPIC full” and “EPIC basic” models in comparison to a state of the art “ABC” model.

FIG. 8C shows performance of EPIC full model in comparison to the ABC model in linking GWAS loci to causal genes.

FIG. 8D shows performance of the “EPIC full” model in comparison to the “ABC” model in HepG2 cells.

FIG. 9A shows the overlapping of E-P pairs and liver-related GWAS loci associations to putative target genes

FIG. 9B further depicts the separate analysis of E-P pairs and GWAS variants and their respective associations with a particular putative target gene.

FIGS. 10A, 10B, and 10C depict three Precision-Recall curves for three additional different versions of the EPIC model in comparison to the ABC model.

### DETAILED DESCRIPTION

### Definitions

The term “obtaining a dataset” encompasses obtaining a set of data determined from at least one sample. Obtaining a dataset encompasses obtaining a sample and processing the sample to experimentally determine the data (e.g., performing one or more assays to determine the data). The phrase also encompasses creating a dataset. The phrase also encompasses receiving a set of data, e.g., from a third party that has processed the sample to experimentally determine the dataset. Additionally, the phrase encompasses mining data from at least one database or at least one publication or a combination of databases and publications. A dataset can be obtained by one of skill in the art via a variety of known ways including stored on a storage memory. In various embodiments, as described herein, a dataset can include one or more of chromatin accessibility data identifying chromatin-accessible regions across the genome and/or chromatin binding data describing chromatin interactions (e.g., chromatin-chromatin interactions, chromatin domains, protein-chromatin binding sites; and transcription factor binding motifs).

The phrase “enhancer-promoter pair” or “E-P pair” refers to an enhancer and a promoter. Methods disclosed herein enable the analysis and characterization of an enhancer-promoter pair to determine whether the enhancer regulates activity of the promoter (and subsequently expression of the gene that is operably linked to the promoter). Furthermore, the phrase “enhancer-gene pair” refers to an enhancer that may influence the expression of the gene. In various embodiments, the phrase “enhancer-gene pair” is used in the context of an “E-P pair” such that the enhancer in the E-P pair may regulate activity of the promoter, thereby regulating expression of the gene in the “enhancer-gene pair.”

The phrases “first set of features” or “basic features” are used interchangeably and refer to features whose values are directly extractable from datasets, such as epigenomic datasets. Example features of the first set of features include, but are not limited to, enhancer or promoter features, chromatin interaction features, and E-P pair distance features.

The phrases “second set of features engineered from subsets of the first set of features,” “engineered features,” and “composite features” are used interchangeably and refer to features whose values are generated by combining values of two or more features of the first set. Example features of the second set of features include, but are not limited to, composite features, gene contribution features, and/or enhancer contribution features.

Any terms not directly defined herein shall be understood to have the meanings commonly associated with them as understood within the art of the disclosure. Certain terms are discussed herein to provide additional guidance to the practitioner in describing the compositions, devices, methods and the like of aspects of the disclosure, and how to make or use them. It will be appreciated that the same thing can be said in more than one way. Consequently, alternative language and synonyms can be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the disclosure herein.

Additionally, as used in the specification, the singular forms “a,” “an” and “the” include plural referents unless the context clearly dictates otherwise.

### Overview

Disclosed herein are methods for characterizing enhancer-promoter pairs, such as characterizing enhancer-promoter pairs as functional or non-functional enhancer-promoter pairs. As used herein, a functional enhancer-promoter pair refers to an enhancer that plays a role in regulating activity or expression of a gene that is operably linked to the promoter. In various embodiments, a functional enhancer-promoter (E-P) pair may include an enhancer that is distally located from the promoter. In particular embodiments, the enhancer may be located up to tens of bases, hundreds of bases, thousands of bases, tens of thousands of bases, hundreds of thousands of bases, or millions of bases away from the location of the promoter.

As disclosed herein, methods for characterizing enhancer-promoter pairs involve the deployment of a machine learning model that incorporates various features. In various embodiments, the machine learning model is trained using training data in which enhancer-promoter pairs are previously determined to be functional or non-functional. As an example, the training data can be generated via a CRISPR-based enhancer perturbation screen in which enhancers are perturbed which may lead to impact on downstream gene expression. Example CRISPR-based enhancer perturbation screens can involve CRISPR interference (CRISPRi) or CRISPR activation (CRISPRa) based perturbation screens. As an example, the training data can be generated via a CRISPRi-based or CRISPRa-based enhancer perturbation screen in which enhancers are perturbed which may lead to impact on downstream gene expression. Example machine learning models incorporate at least a first set of features and a second set of features, herein also referred to as “engineered features” which are generated by combining values of a subset of features of the first set. Altogether, the incorporation of the first set of features and the second set of features enables the machine learning model to more accurately predict whether an E-P pair is a functional or a non-functional E-P pair.

### Regulatory Element Characterization System

Reference is now made to FIG. 1, which depicts a block diagram of an example regulatory element characterization system 100, in accordance with an embodiment. As described herein, the regulatory element characterization system 100 may deploy a machine learning model to predict whether an E-P pair is a functional or a non-functional E-P pair. FIG. 1 is shown to introduce individual components of the regulatory element characterization system 100, examples of which include the epigenomic data module 120, the feature extraction module 130, the model deployment module 140 and the model store 150. In various embodiments, the regulatory element characterization system 100 further includes the model training module 180 and the training data store 190.

Although FIG. 1 shows each of the modules and stores as being present in the regulatory element characterization system 100, in other embodiments, additional or fewer modules and/or stores may be present. For example, as indicated in FIG. 1 by the dotted lines, the model training module 180 and the training data store 190 may, in some embodiments, be operated by another party (e.g., a third party) and are not present in the regulatory element characterization system 100. In such embodiments, the third party may perform the steps of engineering features and training a machine learning model. Thus, the third party may provide the engineered features and the trained machine learning model to the regulatory element characterization system 100 such that the regulatory element characterization system 100 determines values of the engineered features and deploys the machine learning model to predict functional or non-functional E-P pairs.

Referring first to the epigenomic data module 120, it obtains one or more datasets, such as one or more epigenomic datasets. In various embodiments, the epigenomic datasets include chromatin accessibility data (e.g., DNase-seq or ATAC-seq data) and/or chromatin binding data including data describing one or more of DNA-DNA interactions, chromatin domains, protein-chromatin binding sites, and transcription factor binding motifs. Example chromatin binding data includes HiChIP or ChIP-seq data.

In various embodiments, a party that operates the regulatory element characterization system 100 also performs methods and assay for generating the epigenomic datasets. In various embodiments, a different party (e.g., a third party) performs the methods and assay for generating the epigenomic datasets, and provides the epigenomic datasets to the epigenomic data module 120. Example methods and assay for generating ATAC-seq data, HiChIP data, or ChIP-seq data are described in WO2019036430, which is incorporated by reference in its entirety.

Generally, ATAC-seq refers to a process that identifies open chromatin regions and active enhancers. ATAC-seq can include harvesting cells, an example of which includes hepatocytes, and preparing cell nuclei for transposition reactions. In various embodiments, ATAC-seq involves providing a transposase, such as a Tn5 transposase. The transposase can insert an adapter sequence and/or cleave genomic DNA (e.g., at locations of open chromatin regions). Using barcoded primers, nucleic acid amplification can be performed to generate amplicons for sequencing. Using the sequenced reads, ATAC-seq peaks may be called using MACS2 and visualized in the UCSC genome browser. Additional details for performing ATAC-seq are described in Corces et al. (2017) Nat. Methods 14(10): 959-62, which is incorporated by reference in its entirety.

ChIP-seq reveals binding of transcription factors to DNA, modified histones, and chromatin-binding proteins genome wide. Generally, ChIP-seq may first involve cross-linking DNA-protein bound complexes (e.g., genome wide binding between DNA and transcription factors). Samples including the nucleic acids can be fragmented, thereby leaving the DNA-protein bound complexes. Protein specific antibodies, such as antibodies exhibiting binding affinity for particular transcription factors, are provided to immunoprecipitated the DNA-protein complex. The DNA can undergo sequencing to identify the specific DNA sequences that were bound to proteins (e.g., transcription factors). In various embodiments, ChIP-seq further involves a proximity ligation step (e.g., proximity assisted ChIP-seq). Additional details for performing ChIP-seq are described in Johnson et al. (2007) Science 316: 1497-502, which is incorporated by reference in its entirety.

HiChIP is a technique that defines chromatin domains and DNA-DNA interactions, such as enhancer-promoter interactions. HiChIP represents a combination of high-throughputchromosome conformation capture (Hi-C) and chromatin immunoprecipitation sequencing (ChIP-seq). Additional details for performing HiChIP are described in Mumbach et al. (2016) Nat. Methods 13(11): 919-22, which is incorporated by reference in its entirety.

In various embodiments, the chromatin accessibility data represent data that elucidates DNA-protein interaction sites for transcription factors and chromatin binding proteins. For example, the chromatin accessibility data may identify locations on a chromatin occupied by regulatory elements such as an enhancer, repressor, or a promoter. In various embodiments, the chromatin accessibility data are obtained by performing a DNase-seq assay. In various embodiments, the chromatin accessibility data are obtained by performing a Formaldehyde-Assisted Isolation of Regulatory Elements (FAIRE)-seq assay. In various embodiments, the chromatin accessibility data are obtained by performing an Assay for Transposase-Accessible Chromatin using sequencing (ATAC-seq) technique which assesses genome-wide chromatin accessibility. Further details of these techniques for obtaining chromatin accessibility data are described in Tsompana et al. (2014) Epigenetics & Chromatin 7: 33, which is hereby incorporated by reference in its entirety.

DNase-seq is useful for identifying location of regulatory regions in genomic DNA. The methodology is predicated on sequencing of regions that are sensitive to cleavage by DNase I. Generally, DNA-protein complexes are exposed to DNase I. Thus, DNA that are bound to proteins are protected against digestion by DNase I whereas unbound DNA are digested. The DNA bound to protein can then undergo sequencing to identify the sequences that were bound. Additional details for performing DNase-seq are described in Boyle et al. (2008) Cell 132(2): 311-22, which is incorporated by reference in its entirety.

FAIRE-seq is useful for determining sequences in the genome that are associated with regulatory activity. In contrast to DNase-Seq, the FAIRE-Seq protocol doesn’t require the permeabilization of cells or isolation of nuclei. For example, FAIRE-seq can involve crosslinking DNA-protein complexes using formaldehyde, such as nucleosome bound DNA. The cells can be exposed to sonication to fragment the genomic DNA. DNA that is bound to nucleosomes can be separated from unbound DNA through an extraction process (e.g., a phenol-chloroform extraction). Thus, bound DNA remains in the organic phase whereas unbound DNA resides in the aqueous phase. Thus, unbound DNA can be obtained from the aqueous and undergo purification, amplification, and/or sequencing. Additional details for performing FAIRE-seq are described in Giresi et al. (2007) Genome Res. 17(6): 877-85, which is incorporated by reference in its entirety.

In various embodiments, the chromatin accessibility data represent RNA expression data that is generated e.g., via sequencing. In various embodiments, the chromatin accessibility data includes nascent RNA expression data. In various embodiments, the chromatin accessibility data includes Global Run-On Sequencing (GRO-Seq) data, Precision Run-on Sequencing (PRO-seq) data, or PRO-cap data. Here, the RNA expression data may provide insight as to which chromatin regions were available for transcription, resulting in the corresponding RNA expression.

Generally, GRO-seq is useful for measuring RNA, and specifically nascent RNA. GRO-seq involves labelling transcripts with bromouridine (BrU). Furthermore, an anionic surfactant such as sarkosyl is provided which prevents further attachment of RNA polymerase to genomic DNA. This ensures that only new transcripts will be transcribed by RNA polymerase that have already bound to DNA. The labeled RNA transcripts (labeled with BrU) are isolated using an anti-BrdU antibody, reverse transcribed, and sequenced. Additional details for performing GRO-seq are described in Core et al. (2008) Science 322(5909): 1845-8, which is incorporated by reference in its entirety.

PRO-seq is similar to GRO-seq, but can provide additional single-base resolution information. Generally, PRO-seq involves a run-on reaction with biotin-NTPs and an anionic surfactant such as sarkosyl. The anionic surfactant prevents further attachment of RNA polymerase to genomic DNA. Furthermore, the incorporation of the biotin-NTPs prevents the elongation of RNA transcripts. The RNA transcripts can be extracted and can further undergo purification (e.g., using streptavidin pull down). RNA transcripts undergo reverse transcription, amplification, and sequencing. Additional details for performing PRO-seq are described in Mahat (2016) Nat. Protoc. 11(8): 1455-76, which is incorporated by reference in its entirety.

In various embodiments, the chromatin binding data describes chromatin domains and chromatin interactions (e.g., DNA-DNA interactions). DNA-DNA interactions, in some embodiments, are mediated by SMC1A, CTCF and H3K27Ac. The chromatin binding data enables profiling of three-dimensional chromatin structures. In various embodiments, one or more of the DNA-DNA interactions described in the chromatin binding data are enhancer-promoter interactions. In various embodiments, one or more of the chromatin domains described in the chromatin binding data are insulated neighborhoods. In various embodiments, the chromatin binding data are obtained by performing HiChIP followed by sequencing (e.g., next generation sequencing). In various embodiments, HiChIP includes performing paired-end-tag (PET) sequencing for ultra-high-throughput sequencing. Additional details for performing PET sequencing are described in Fullwood et al. (2010) Curr. Protoc. Mol. Biol. Chapter 21: Unit 21.15.1-25, which is incorporated by reference in its entirety.

In various embodiments, the protein-chromatin binding site data represent data that reveal binding sites of transcription factors as well as the proteins that bind to those particular binding sites. Example proteins can include any of H3K27ac, H3K4me1, H3K4me3, BRD4, EP300, MED1, Pol2, YY1, RAD21, CTCF, H3K27me3, H3K9me3, H4K20me1, NCOR1, HDAC1/2/3, EZH2, SUZ12, ZEB2, and REST. More generally, example proteins can include transcription factors, active enhancer marks (e.g., H3K27ac or H3K4me1), and repressive factors (e.g., H3K27me3, H3K9me3, H4K20me1, NCOR1, HDAC1/2/3, EZH2, SUZ12, ZEB2, REST). The protein-chromatin binding site data may further include data that reveal histone modifications. In various embodiments, the protein-chromatin binding site data are obtained by performing Chromatin Immunoprecipitation (ChIP) followed by sequencing (e.g., next generation sequencing). In various embodiments, the DNA-binding motif data represent data that describes particular DNA sequences. For example, the DNA-binding motif data may describe transcription factor binding motifs (e.g., specific DNA sequences where transcription factors bind to the chromatin). In various embodiments, the chromatin motif is obtained through publicly available databases (e.g., Human Transcription Factor motifs database).

In various embodiments, the epigenomic data module 120 identifies E-P pairs in the epigenomic datasets. The epigenomic data module 120 may identify E-P pairs in which the enhancer and the promoter are within a genomic distance threshold. In various embodiments, the epigenomic data module 120 may identify all E-P pairs within a 10 Mb genomic distance. In various embodiments, the epigenomic data module 120 may identify all E-P pairs within a 9 Mb, 8 Mb, 7 Mb, 6 Mb, 5 Mb, 4 Mb, 3 Mb, 2 Mb, 1 Mb, 0.9 Mb, 0.8 Mb, 0.7 Mb, 0.6 Mb, 0.5 Mb, 0.4 Mb, 0.3 Mb, 0.2 Mb, or 0.1 Mb genomic distance.

In various embodiments, the epigenomic data module 120 identifies presence of enhancers or promoters based on peaks present in the epigenomic data. In some embodiments, the epigenomic data module 120 identifies presence of enhancers or promoters based on peaks present in chromatin-accessibility data in the form of ATAC-seq data and protein-chromatin binding site data. The epigenomic data module 120 may identify enhancer candidates as the union of one or more peaks in the epigenomic datasets. In some embodiments, an enhancer is represented by presence of a peak in the chromatin accessibility data (e.g., ATAC-seq). In some embodiments, an enhancer is characterized by histone modifications high in H3K4me1 and H3K27ac. In various embodiments, an enhancer is characterized by a presence of low H3K4me3 histone modifications (e.g., as evidenced by a low peak or lack of a peak in protein-chromatin binding site data). In particular embodiments, the epigenomic data module 120 identifies an enhancer as the union of EP300 ChIP-seq peaks and the peaks of ATAC-seq data that overlap with H3K27ac or H3K4me1 ChIP-seq peaks.

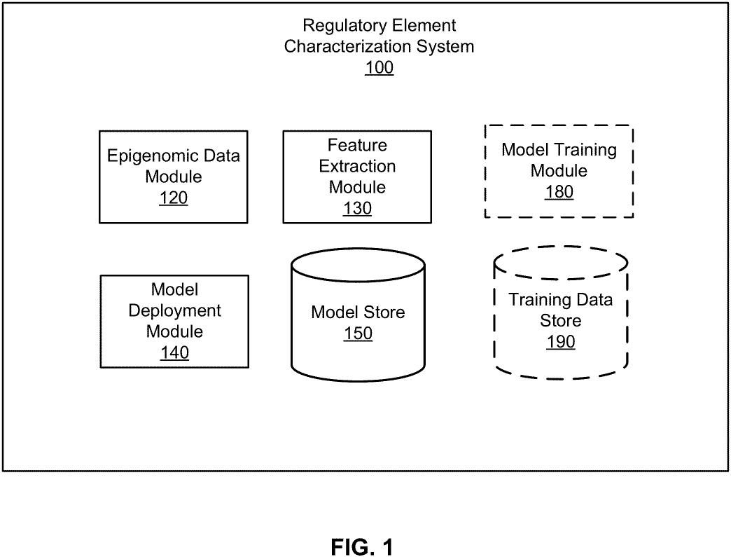

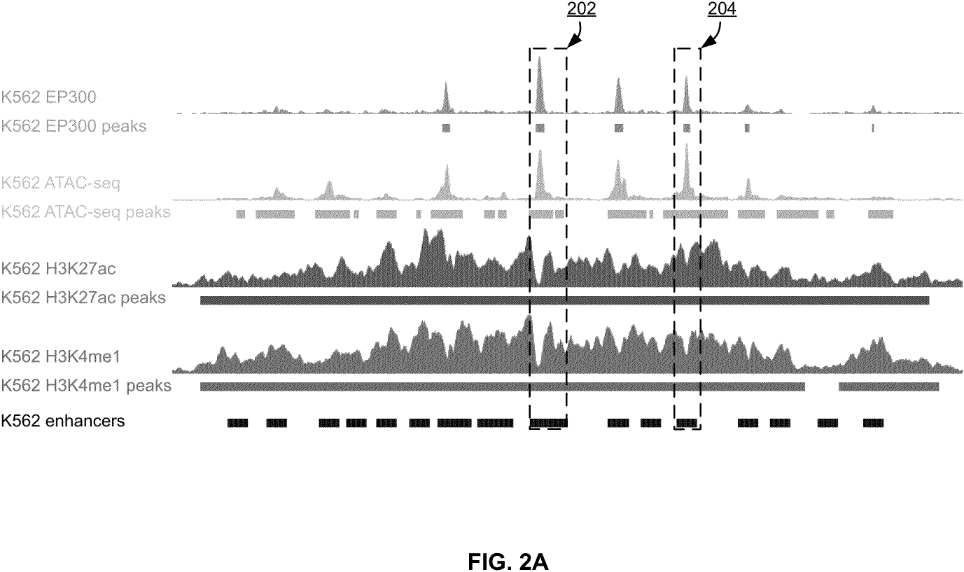

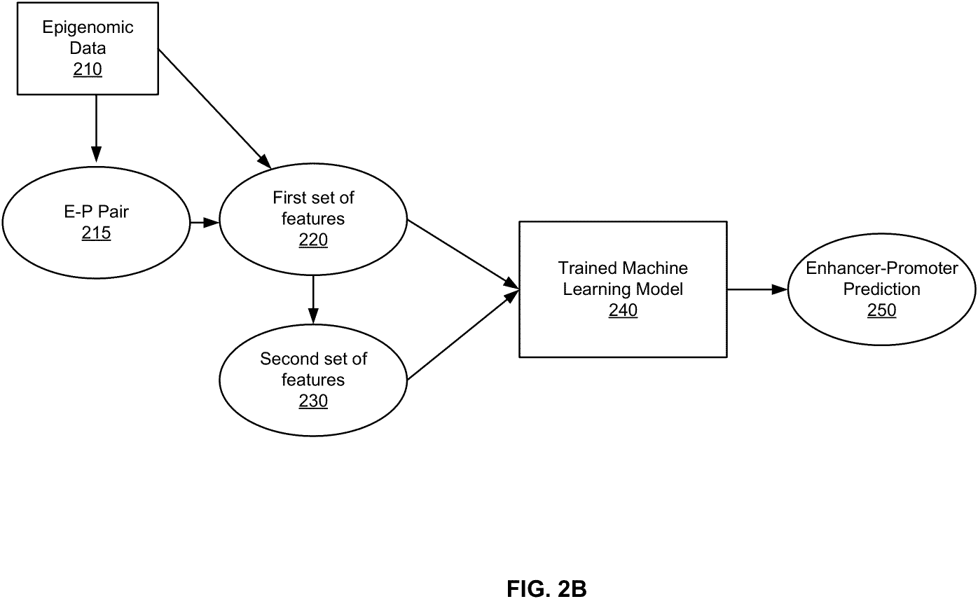

Reference is now made to FIG. 2A, which depicts example data (e.g., EP300, ATAC-seq, H3K27ac, K3K4me1) of an epigenomic dataset, in accordance with an embodiment. Here, the epigenomic data module 120 can use the example data to identify an enhancer. In particular, FIG. 2A shows EP300 data, ATAC-seq data, H3K27ac data, and H3K4me1 data. Here, the epigenomic data module 120 can identify at least genomic region 202 and genomic region 204 as enhancers based on the peaks in the EP300, ATAC-seq, H3K27ac, and/or H3K4me1 data. Specifically, referring to genomic region 202, first the union of the EP300 peak and the ATAC-seq peak is identified. Although the EP300 peak does not span the full genomic region 202, the union of the EP300 peak and the ATAC-seq peak spans the full genomic region 202. Furthermore, the union of the EP300 peak and the ATAC-seq peak is compared to either the H3K27ac peak or the H3K4me1 peak. Here, both the H3K27ac peak and the H3K4me1 peak span the full genomic region 202. Thus, given the overlap between the union of the EP300 peak and the ATAC-seq peak and either the H3K27ac peak or the H3K4me1 peak, the epigenomic data module 120 identifies the genomic region 202 as an enhancer. The epigenomic data module 120 may perform a similar analysis to identify genomic region 204 as an enhancer.

In various embodiments, the epigenomic data module 120 identifies promoter regions which represent the transcription start site of protein-coding and lncRNA genes (GENCODE v24).

Referring to the feature extraction module 130, it extracts values of features from the epigenomic datasets for the identified E-P pairs. In particular embodiments, the feature extraction module 130 extracts values of features directly from the epigenomic datasets (also referred to herein as the first set of features). Examples of the first set of features are described in further detail herein. In various embodiments, the feature extraction module 130 further determines values of a second set of features (also referred to herein as the second set of features). Here, the feature extraction module 130 combines values of features of the first set to generate values of the second set of features. Further details of the second set of features as well as example methods for determining values of the second set of features from the first set of features are described herein.

The model deployment module 140 accesses a trained machine learning model from the model store 150 and deploys the trained machine learning model. In particular embodiments, the model deployment module 140 deploys the trained machine learning model to analyze the features of an E-P pair (including first set and second set of features of an E-P pair). Thus, the trained machine learning model outputs a prediction that is informative as to whether the E-P pair is a functional E-P pair or a non-functional E-P pair. For example, the trained machine learning model outputs a score that is informative as to whether the E-P pair is a functional E-P pair or a non-functional E-P pair. In various embodiments, the score is compared to a threshold value to determine whether the E-P pair is a functional E-P pair or a non-functional E-P pair. For example, in one embodiment, if the score outputted by the trained machine learning model is below the threshold value, the E-P pair is deemed a functional E-P pair. If the score outputted by the trained machine learning model is above the threshold value, the E-P pair is deemed a non-functional E-P pair. In some embodiments, if the score outputted by the trained machine learning model is below the threshold value, the E-P pair is deemed a non-functional E-P pair. If the score outputted by the trained machine learning model is above the threshold value, the E-P pair is deemed a functional E-P pair.

In various embodiments, a selected threshold value is a value between 0 and 1. In various embodiments, the selected threshold value is 0.1, 0.2, 0.3, 0.4, 0.5, 0.6, 0.7, 0.8, or 0.9. In particular embodiments, the selected threshold value is 0.5. In particular embodiments, if the score outputted by the trained machine learning model is greater than (or greater than or equal to) the threshold value of 0.5, the E-P pair is deemed a functional E-P pair. In particular embodiments, if the score outputted by the trained machine learning model is below the threshold value of 0.5, the E-P pair is deemed a non-functional E-P pair. In particular embodiments, the selected threshold value is 0.4. In particular embodiments, if the score outputted by the trained machine learning model is greater than (or greater than or equal to) the threshold value of 0.4, the E-P pair is deemed a functional E-P pair. In particular embodiments, if the score outputted by the trained machine learning model is below the threshold value of 0.4, the E-P pair is deemed a non-functional E-P pair.

The trained machine learning model may be iteratively applied to analyze features of additional E-P pairs. Thus, the trained machine learning model can predict whether each of the additional E-P pairs are functional or non-functional E-P pairs. The predicted functional enhancer-promoter pairs may be useful e.g., for development of novel therapeutics that target enhancers of disease-related genes.

The model training module 180 performs the training of the machine learning models using training data obtained from the training data store 190. Generally, the model training module 180 trains the machine learning model such that the machine learning model is able to better distinguish between functional E-P pairs and non-functional E-P pairs. In various embodiments, the model training module 180 trains the machine learning model using supervised training techniques. For example, the training data may include labels as to whether certain E-P pairs in the training data (also referred to as “training E-P pairs”) are functional or non-functional E-P pairs. Therefore, through supervised training, the machine learning model learns to distinguish between features of functional E-P pairs and non-functional E-P pairs according to the labels of the training data.

In various embodiments, the training data include epigenomic datasets for the training E-P pairs. Thus, values of features, such as a first set of features and a second set of features engineered from combinations of features of the first set, can be determined for each of the training E-P pairs. Together, the values of features for a training E-P pair as well as the indication as to whether the training E-P pair is a functional or non-functional E-P pair, can be referred to as a training example. In various embodiments, the model training module 180 iteratively trains the machine learning model across the training examples. At each iteration, the machine learning model predicts whether the training E-P pair is a functional or non-functional E-P pair. The prediction of the machine learning model is compared to the label and the parameters of the machine learning model are adjusted to improve the predictions outputted by the machine learning model.

In various embodiments, the training data used to train the machine learning model can be derived from a particular cell type. For example, the training data used to train the machine learning model may be derived from a cancer cell line (e.g., HepG2, HEK293, HeLa, or K562 cells). In particular embodiments, the training data used to train the machine learning model may be derived from a leukemia cell line. In particular embodiments, the training data used to train the machine learning model may be derived from K562 cells. In various embodiments, the machine learning model is trained on a first cell type, and the machine learning model is deployed (e.g., deployed by the model deployment module 140) to analyze E-P pairs of epigenomic datasets derived from other cell types. In various embodiments, other cell types can be cells derived from an organ, examples of which include any of the brain, heart, eye, thorax, lung, abdomen, colon, cervix, pancreas, kidney, liver, muscle, lymph nodes, esophagus, intestine, spleen, stomach, skin, bone, and gall bladder.

In various embodiments, the training data used to train the machine learning model is generated via a screen that reveals whether certain enhancers influence the expression of certain genes. In particular embodiments, the training data are generated using an enhancer perturbation screen in which enhancers are perturbed. The expression of certain genes prior to and subsequent to the perturbation of the enhancers can be investigated to determine whether the enhancer plays a role in regulating the expression of the gene. Thus, an enhancer that influences the expression of a gene can be labeled as a functional E-P pair, where the gene is operably linked to the promoter of the functional E-P pair. In contrast, an enhancer that does not influence the expression of a gene can be labeled as a non-functional E-P pair, where the gene is operably linked to the promoter of the non-functional E-P pair. In particular embodiments, the enhancer perturbation screen is a CRISPRi-based enhancer perturbation screen. In particular embodiments, the enhancer perturbation screen is a CRISPRa-based enhancer perturbation screen.

Following training, the model training module 180 locks the parameters of the machine learning model, and stores the machine learning models in the model store 150 for subsequent retrieval and deployment by the model deployment module 140.

### Methods for Determining Functional Enhancer-Promoter Pairs

FIG. 2B depicts a flow diagram for characterizing regulatory elements, in accordance with an embodiment. FIG. 2B begins with obtaining epigenomic data 210, examples of which include one or more of HiC/HiChIP data, ChIP-seq data, DNase-seq data, ATAC-seq data, PRO-seq data, and PRO-cap data.

As shown in FIG. 2B, the epigenomic data 210 are used to define an E-P pair. For example, all E-P pairs within certain genomic distance thresholds are identified. Methods for identifying enhancers and promoters are described herein.

For each E-P pair, values for a first set of features 220 for the E-P pair are extracted from the epigenomic data. This step may be performed by the feature extraction module 130, as described in relation to FIG. 1. Examples of features of the first set include enhancer or promoter features, chromatin interaction features, and E-P pair distance features (e.g., the distance between the enhancer and promoter of the E-P pair). Features of the first set are described in further detail herein. In various embodiments, the feature extraction module 130 extracts values for at least five features of the first set of features 220. In various embodiments, the feature extraction module 130 extracts values for at least ten features, at least fifteen features, at least twenty features, at least twenty five features, at least thirty features, at least thirty five features, at least forty features, at least forty five features, at least fifty features, at least sixty features, at least sixty five features, at least seventy features, at least seventy five features, at least eighty features, at least eighty five features, at least ninety features, at least ninety five features, or at least one hundred features of the first set of features 220. In particular embodiments, the feature extraction module 130 extracts values for at least seventy features of the first set of features 220. In particular embodiments, the feature extraction module 130 extracts values for seventy-five features of the first set of features 220.

The feature extraction module 130 may further generate values for a second set of features 230 derived from a subset of features of the first set of features 220. Features of the second set are described in further detail herein. In various embodiments, the feature extraction module 130 generates at least one feature of the second set by combining a subset of features from the first set. In various embodiments, the feature extraction module 130 generates at least two features, at least three features, at least four features, at least five features, at least six features, at least seven features, at least eight features, at least nine features, or at least ten features of the second set by combining a subset of features from the first set. In particular embodiments, the feature extraction module 130 generates nine features of the second set by combining a subset of features from the first set. In particular embodiments, the feature extraction module 130 generates nine features of the second set by combining a subset of features from the first set. In particular embodiments, the feature extraction module 130 generates ten features of the second set by combining a subset of features from the first set.

The first set of features 220 and the second set of features 230 of an E-P pair are provided as input to the trained machine learning model 240. The trained machine learning model 240 analyzes the first set of features 220 and the second set of features 230 to generate an enhancer-promoter prediction 250. For example, the enhancer-promoter prediction 250 can be an identification of a functional or a non-functional E-P pair. Generally, by analyzing both the first set of features 220 and second set of features 230 for an E-P pair, the trained machine learning model is able to better predict whether the E-P pair is functional or non-functional in comparison to a machine learning model that analyzes solely the first set of features 220.

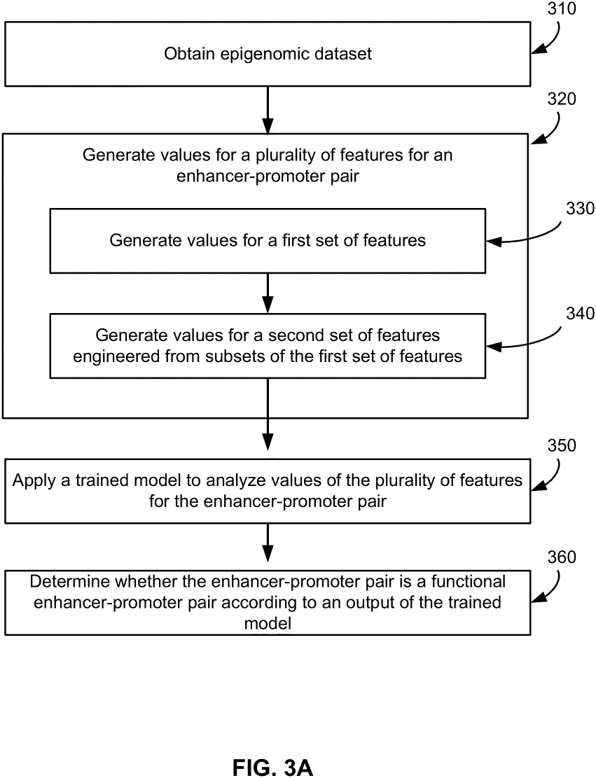

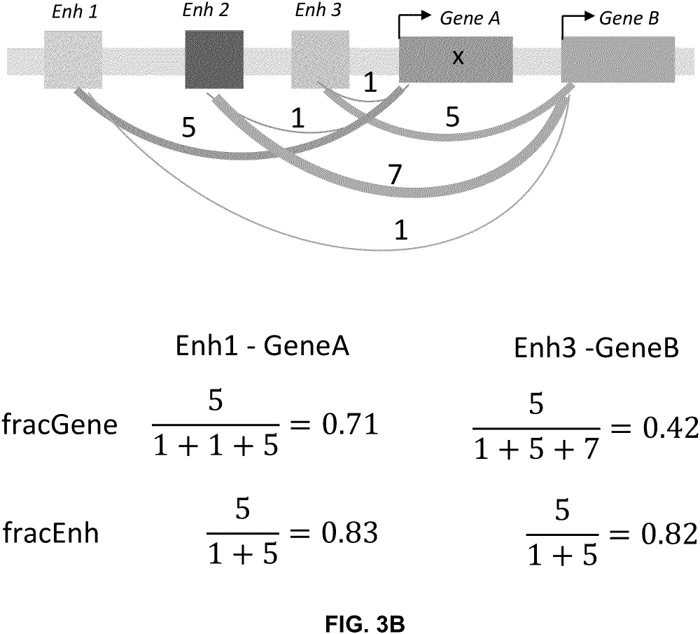

FIG. 3A is a flow process for characterizing regulatory elements, in accordance with an embodiment. Step 310 involves obtaining one or more epigenomic datasets. In various embodiments, the epigenomic datasets include chromatin accessibility data (e.g., DNase-seq or ATAC-seq data) and/or chromatin binding data including data describing one or more of DNA-DNA interactions, chromatin domains, protein-chromatin binding sites, and transcription factor binding motifs. Example chromatin binding data includes HiChIP or ChIP-seq data.

Step 320 involves generating values for a plurality of features for an enhancer-promoter pair. As shown in FIG. 3A, step 320 may include both steps 330 and 340. Step 330 involves generating values for a first set of features. Values of the first set of features may be extracted directly from the one or more epigenomic datasets. Example features of the first set may include enhancer/promoter features, chromatin interaction features, and/or E-P pair distance features.

Step 340 involves generating values for a second set of features that are engineered from subsets of the first set of features. Here, features of the second set represent more complex features that may be more informative for predicting functional enhancer-promoter pairs in comparison to solely the features of the first set. As described herein, example features of the second set include, but are not limited to: APMI, fracEnh, fracGene, fracGmE, fracGpE, apmiGene, apmiEnh, apmiGmE, and apmiGpE.

Step 350 involves applying a trained model to analyze values of the plurality of features for the enhancer-promoter pair. For example, the values of the first set of features and the values of the second set of features are provided as input to the trained machine learning model.

Step 360 involves determining whether the enhancer-promoter pair is a functional enhancer-promoter pair according to an output of the trained model. A predicted functional enhancer-promoter pair may be useful e.g., for development of novel therapeutics that target enhancers of disease-related genes.

Although not explicitly shown in FIG. 3A, the steps of 320-360 can be repeated for additional enhancer-promoter pairs for which data are present in the one or more epigenomic datasets. Therefore, additional enhancer-promoter pairs can be analyzed to determine whether each of the additional enhancer-promoter pairs is a functional or non-functional enhancer-promoter pair.

### Example Features and Engineered Features for Predicting Functional Enhancer Promoter Pairs

As disclosed herein, machine learning models may include different features for predicting functional enhancer-promoter pairs. In various embodiments, a machine learning model includes a first set of features, wherein values of the first set of features can be directly extracted from epigenomic datasets. In various embodiments, a machine learning model includes a second set of features (also referred to herein as “engineered features”), wherein values of the second set of features are generated by combining values of subsets of features from the first set of features.

Example features of the first set of features include, but are not limited to, genomic distance, enhancer or promoter features, chromatin interaction features, and E-P pair distance features (e.g., the distance between the enhancer and promoter of the E-P pair). Example enhancer or promoter features include ChIP-seq features of H3K27ac, H3K4me1, H3K4me3, BRD4, EP300, MED1, Pol2, YY1, RAD21, CTCF, and Input. Further examples of enhancer or promoter features include ChIP-seq features of repressive factors including H3K27me3, H3K9me3, H4K20me1, NCOR1, HDAC1/2/3, EZH2, SUZ12, ZEB2, and REST. Additional examples of enhancer or promoter features can refer to characteristics from any of ATAC-seq, DNase-seq, PRO-seq, or PRO-cap (hereafter referred to as ATAC features, DNase features, PRO-seq features, or PRO-cap features).

In various embodiments, the enhancer or promoter features may include read counts in a variety of different window sizes. In various embodiments, a window size can refer to any of ± 50 bp, ± 100 bp, ± 150 bp, ± 200 bp, ± 250 bp, ± 300 bp, ± 350 bp, ± 400 bp, ± 450 bp, ± 500 bp, ± 600 bp, ± 700 bp, ± 800 bp, ± 900 bp, ± 1000 bp, ± 1100 bp, ± 1200 bp, ± 1300 bp, ± 1400 bp, ± 1500 bp, ± 1600 bp, ± 1700 bp, ± 1800 bp, ± 1900 bp, ± 2000 bp, ± 2100 bp, ± 2200 bp, ± 2300 bp, ± 2400 bp, and ± 2500 bp. For example, different ATAC features can include ATAC features within a first window size (e.g., ± 150 bp), ATAC features within a second window size (e.g., ± 250 bp), ATAC features within a third window (e.g., ± 500 bp), ATAC features within a fourth window (e.g., ± 1000 bp), and ATAC features within a fifth window (e.g., ± 2000 bp). ChIP-seq features, DNase-seq features, PRO-seq features, and PRO-cap features may be similarly generated according to multiple window sizes.

Chromatin interaction features can include features that describe chromatin binding. Example chromatin interaction features include K3K27ac HiChIP features. In various embodiments, the chromatin interaction features may include paired-end read counts in a variety of different window sizes. In various embodiments, a window size can refer to any of ± 500 bp, ± 1000 bp, ± 1500 bp, ± 2000 bp, ± 2500 bp, ± 3000 bp, ± 3500 bp, ± 4000 bp, ± 4500 bp, ± 5000 bp, ± 5500 bp, ± 6000 bp, ± 6500 bp, ± 7000 bp, ± 7500 bp, ± 8000 bp, ± 8500 bp, ± 9000 bp, ± 9500 bp, ± 10000 bp, ± 11000 bp, ± 12000 bp, ± 13000 bp, ± 14000 bp, ± 15000 bp, ± 16000 bp, ± 17000 bp, ± 18000 bp, ± 19000 bp, ± 20000 bp, ± 21000 bp, ± 22000 bp, ± 23000 bp, ± 24000 bp, or ± 25000 bp. For example, chromatin interaction features can include different chromatin interactions features within a first window (e.g., ± 2500 bp), different chromatin interaction features within a second window (e.g., ± 5000 bp), different chromatin interaction features within a second window (e.g., ± 7500 bp), and different chromatin interaction features within a second window (e.g., ± 10000 bp).

In various embodiments, machine learning models disclosed herein include one or more features from the first set of features, wherein values of the first set of features can be directly extracted from epigenomic datasets. In various embodiments, machine learning models disclosed herein include at least 2, at least 3, at least 4, a least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 features of the first set of features. In particular embodiments, machine learning models disclosed herein include 10, 20, 30, 40, 50, 60, 70, 80, 90, or 100 features of the first set of features. In particular embodiments, machine learning models disclosed herein include 75 features of the first set of features.

In particular embodiments, features of the first set of models of the machine learning model include a genomic distance feature (e.g., genomic distance between an enhancer and promoter). In particular embodiments, features of the first set of models of the machine learning model include HiChIP features of 4 or more different window sizes. For example, features of the first set can include HiChIP features within window sizes of 5 kb, 10 kb, 15 kb, and/or 20 kb. In particular embodiments, features of the first set of models of the machine learning model include ChIP-seq features. For example, features of the first set can include one or more of H3K27ac, H3K4me1, H3K4me3, EP300, CTCF, and Input features. As another example, features of the first set can include two or more, three or more, four or more, five or more, or each of H3K27ac, H3K4me1, H3K4me3, EP300, CTCF, and Input features. In particular embodiments, features of the first set of models of the machine learning model include ATAC-seq features. In particular embodiments, features of the first set of models of the machine learning model include ChIP-seq features and/or ATAC-seq features across five or more windows. For example, features of the first set can include ChIP-seq features and/or ATAC-seq features within window sizes of 300bp, 500bp, 1 kb, 2 kb, or 4 kb.

Features of the second set of features are generated by combining subsets of the features of the first set. In various embodiments, features of the second set include one or more composite features representing a combination of features of the first set. In various embodiments, a composite feature represents a combination of two or more, three or more, four or more, five or more, six or more, seven or more, eight or more, nine or more, or ten or more features of the first set. In particular embodiments, a composite feature represents a combination of two or more features of the first set. In particular embodiments, a composite feature represents a combination of three or more features of the first set. In particular embodiments, a composite feature represents a combination of four or more features of the first set. For example, a composite feature represents a combination of 1) an ATAC feature, 2) a EP300 feature, 3) a H3K4me1 feature, and 4) a HiChIP feature. As a specific example, a composite feature, represented as “APMI” can be denoted as:

$$A P M I = {\left(A T A C ∗ E P 300 ∗ H 3 K 4 m e 1\right)}^{\frac{1}{3}} ∗ H i C h I P$$

In various embodiments, features of the second set include an enhancer contribution feature, which represents a quantified relative contribution of an enhancer in the E-P pair to a gene, wherein the gene is operably linked to the promoter of the E-P pair. Put another way, the enhancer contribution feature quantifies the strength of the contribution by the enhancer of the E-P pair to the gene in comparison to other genes that are linked to the enhancer. In various embodiments, the enhancer contribution feature is generated by combining subsets of features of the first set. In various embodiments, the enhancer contribution feature is generated using one or more composite features (e.g., referred to above as “APMI”). In various embodiments, the enhancer contribution feature is a ratio of the composite feature of the enhancer to a combination of a plurality of composite features for the enhancer. As a specific example, the relative contribution of a particular enhancer e to a gene g from the enhancer perspective is represented as:

$$f r a c E n {h}_{e g} = \frac{A P M {I}_{e g}}{{\sum }_{k} A P M {I}_{e k}}$$

where k indexes all the genes connected to enhancer e.

In various embodiments, features of the second set include a gene contribution feature, which represents a quantified relative contribution of an enhancer in the E-P pair to a gene, wherein the gene is operably linked to the promoter of the E-P pair. Put another way, the gene contribution feature quantifies the strength of the contribution by the enhancer of the E-P pair to the gene in comparison to other enhancers that are linked to the gene. In various embodiments, the enhancer contribution feature is generated by combining subsets of features of the first set. In various embodiments, the enhancer contribution feature is generated using one or more composite features (e.g., referred to above as “APMI”). In various embodiments, the enhancer contribution feature is a ratio of the composite feature of the gene to a combination of a plurality of composite features for the gene. As a specific example, the relative contribution of a particular enhancer e to a gene g from the gene perspective is represented as:

$$f r a c G e n {e}_{e g} = \frac{A P M {I}_{e g}}{{\sum }_{j} A P M {I}_{j g}}$$

where j indexes all the enhancers connected to gene g.

FIG. 3B shows example determination of values of engineered features, in accordance with an embodiment. Here, FIG. 3B shows the determination of values for the fracGene and fracEnh features for pairs of enhancers and genes (e.g., genes operably linked to a promoter of the E-P pair). FIG. 3B shows the presence of an enhancer 1 (Enh 1), enhancer 2 (Enh 2), enhancer 3 (Enh 3), a Gene A, and a Gene B. Furthermore, FIG. 3B shows example APMI values of Enhancer-Gene pairs. For example, the APMI value between Enh 1 and Gene A is 5, the APMI value between Enh 1 and Gene B is 1, and so on.

The fracGene feature for Enh1-GeneA, which represents the relative contribution of Enhancer 1 to Gene A from the gene perspective, is denoted as fracGene_{Enh1-GeneA} and can be calculated as:

$$\frac{A P M {I}_{E n h 1 \text{−} G e n e A}}{A P M {I}_{E n h 1 \text{−} G e n e A} + A P M {I}_{E n h 2 \text{−} G e n e A} + A P M {I}_{E n h 3 \text{−} G e n e A}}$$

Thus, the value of the relative contribution of Enhancer 1 to Gene A from the gene perspective can be determined to be

$$\frac{5}{1 + 1 + 5} = 0.71.$$

Additionally, the fracEnh feature for Enh1-GeneA, which represents the relative contribution of Enhancer 1 to Gene A from the enhancer perspective, is denoted as fracEnh_{Enh1-GeneA} and can be calculated as:

$$\frac{A P M {I}_{E n h 1 \text{−} G e n e A}}{A P M {I}_{E n h 1 \text{−} G e n e A} + A P M {I}_{E n h 1 \text{−} G e n e B}}$$

Thus, the value of the relative contribution of Enhancer 1 to Gene A from the enhancer perspective can be determined to be

$$\frac{5}{1 + 5} = 0.83.$$

As shown in FIG. 3B, the fracGene and fracEnh feature values can also be calculated for Enh3-Gene B. Although not shown in FIG. 3B, additional fracGene and fracEnh feature values can be calculated for other enhancer gene pairs (e.g., Enh1-Gene B, Enh2-Gene A, Enh2-Gene B, and Enh3-Gene A).

In various embodiments, features of the second set further include additional engineered features. In various embodiments, additional engineered features are generated by combining subsets of features of the first subset. In various embodiments, additional engineered features are generated by combining composite features. In various embodiments, additional engineered features are generated by combining gene contribution features and enhancer contribution features. For example, two example additional engineered features representing combinations of gene contribution features and enhancer contribution features can be denoted as:

$$\begin{matrix}f r a c G m {E}_{e g} = f r a c G e n {e}_{e g} ∗ f r a c E n {h}_{e g} \\ f r a c G p {E}_{e g} = f r a c G e n {e}_{e g} ∗ f r a c E n {h}_{e g}\end{matrix}$$

In various embodiments, additional engineered features are generated by combining composite features with gene contribution features. For example, an example additional engineered feature representing combinations of composite features and gene contribution features can be denoted as:

$$a p m i G e n {e}_{e g} = f r a c G e n {e}_{e g} ∗ A P M {I}_{e g}$$

In various embodiments, additional engineered features are generated by combining composite features with enhancer contribution features. For example, an example additional engineered feature representing combinations of composite features and enhancer contribution features can be denoted as:

$$a p m i E n {h}_{e g} = f r a c E n {h}_{e g} ∗ A P M {I}_{e g}$$

In various embodiments, the second set of features include yet additional engineered features. As an example, yet additional engineered features are generated by combining composite features and additional engineered features. Examples of yet additional engineered features can be denoted as:

$$a p m i G m {E}_{e g} = f r a c G m {E}_{e g} ∗ A P M {I}_{e g}$$

$$a p m i G p {E}_{e g} = f r a c G p {E}_{e g} ∗ A P M {I}_{e g}$$

In various embodiments, machine learning models disclosed herein include both a first set of features, wherein values of the first set of features can be directly extracted from epigenomic datasets, and a second set of features, wherein values of the second set of features are generated by combining values of subsets of features from the first set of features. In various embodiments, machine learning models disclosed herein include at least 1 engineered feature of the second set of features. In various embodiments, machine learning models disclosed herein include at least 2, at least 3, at least 4, a least 5, at least 6, at least 7, at least 8, at least 9, at least 10, at least 15, at least 20, at least 25, at least 30, at least 40, at least 50, at least 60, at least 70, at least 80, at least 90, or at least 100 engineered features of the second set of features. In particular embodiments, machine learning models disclosed herein include 1, 2, 3, 4, 5, 6, 7, 8, or 9 engineered features.

In various embodiments, machine models disclosed herein include 1 engineered feature selected from APMI, fracEnh, fracGene, fracGmE, fracGpE, apmiGene, apmiEnh, apmiGmE, or apmiGpE. In various embodiments, machine models disclosed herein include 2 engineered features, such as APMI and fracEnh, APMI and fracGene, APMI and fracGmE, APMI and fracGpE, APMI and apmiGene, APMI and apmiEnh, APMI and apmiGmE, APMI and apmiGpE, fracEnh and fracGene, fracEnh and fracGmE, fracEnh and fracGpE, fracEnh and apmiGene, fracEnh and apmiEnh, fracEnh and apmiGmE, fracEnh and apmiGpE, fracGene and fracGmE, fracGene and fracGpE, fracGene and apmiGene, fracGene and apmiGmE, fracGene and apmiGpE, fracGmE and fracGpE, fracGmE and apmiGene, fracGmE and apmiEnh, fracGmE and apmiGmE, fracGmE and apmiGpE, fracGpE and apmiGene, fracGpE and apmiEnh, fracGpE and apmiGmE, fracGpE and apmiGpE, apmiGene and apmiEnH, apmiGene and apmiGmE, apmiGene and apmiGpE, apmiEnh and apmiGmE, apmiEnh and apmiGpE, and apmiGmE and apmiGpE.

In various embodiments, machine models disclosed herein include 3 engineered features, including combinations such as 1) APMI, fracEnh, fracGene, 2) APMI, fracEnh, fracGmE, 3) APMI, fracEnh, fracGpE, 4) APMI, fracEnh, apmiGene, 5) APMI, fracEnh, apmiEnh, 6) APMI, fracEnh, apmiGmE, 7) APMI, fracEnh, apmiGpE, 8) APMI, fracGene, fracGmE, 9) APMI, fracGene, fracGpE, 10) APMI, fracGene, apmiGene, 11) APMI, fracGene, apmiEnh, 12) APMI, fracGene, apmiGmE, 13) APMI, fracGene, apmiGpE, 14) APMI, fracGmE, fracGpE, 15) APMI, fracGmE, apmiGene, 16) APMI, fracGmE, apmiEnh, 17) APMI, fracGmE, apmiGmE, 18) APMI, fracGmE, apmiGpE, 19) APMI, fracGpE, apmiGene, 20) APMI, fracGpE, apmiEnh, 21) APMI, fracGpE, apmiGmE, 22) APMI, fracGpE, apmiGpE, 23) APMI, apmiGene, apmiEnh, 24) APMI, apmiGene, apmiGmE, 25) APMI, apmiGene, apmiGpE, 26) APMI, apmiEnh, apmiGmE, 27) APMI, apmiEnh, apmiGpE, 28) APMI, apmiGmE, apmiGpE, 29) fracEnh, fracGene, fracGmE, 30) fracEnh, fracGene, fracGpE, 31) fracEnh, fracGene, apmiGene, 32) fracEnh, fracGene, apmiEnh, 33) fracEnh, fracGene, apmiGmE, 34) fracEnh, fracGene, apmiGpE, 35) fracEnh, fracGmE, fracGpE, 36) fracEnh, fracGmE, apmiGene, 37) fracEnh, fracGmE, apmiEnh, 38) fracEnh, fracGmE, apmiGmE, 39) fracEnh, fracGmE, apmiGpE, 40) fracEnh, fracGpE, apmiGene, 41) fracEnh, fracGpE, apmiEnh, 42) fracEnh, fracGpE, apmiGmE, 43) fracEnh, fracGpE, apmiGpE, 44) fracEnh, apmiGene, apmiEnh, 45) fracEnh, apmiGene, apmiGmE, 46) fracEnh, apmiGene, apmiGpE, 47) fracEnh, apmiEnh, apmiGmE, 48) fracEnh, apmiEnh, apmiGpE, 49) fracEnh, apmiGmE, apmiGpE, 50) fracGene, fracGmE, fracGpE, 51) fracGene, fracGmE, apmiGene, 52) fracGene, fracGmE, apmiEnh, 53) fracGene, fracGmE, apmiGmE, 54) fracGene, fracGmE, apmiGpE, 55) fracGene, fracGpE, apmiGene, 56) fracGene, fracGpE, apmiEnh, 57) fracGene, fracGpE, apmiGmE, 58) fracGene, fracGpE, apmiGpE, 59) fracGene, apmiGene, apmiEnh, 60) fracGene, apmiGene, apmiGmE, 61) fracGene, apmiGene, apmiGpE, 62) fracGene, apmiEnh, apmiGmE, 63) fracGene, apmiEnh, apmiGpE, 64) fracGene, apmiGmE, apmiGpE, 65) fracGmE, fracGpE, apmiGene, 66) fracGmE, fracGpE, apmiEnh, 67) fracGmE, fracGpE, apmiGmE, 68) fracGmE, fracGpE, apmiGpE, 69) fracGmE, apmiGene, apmiEnh, 70) fracGmE, apmiGene, apmiGmE, 71) fracGmE, apmiGene, apmiGpE, 72) fracGmE, apmiEnh, apmiGmE, 73) fracGmE, apmiEnh, apmiGpE, 74) fracGmE, apmiGmE, apmiGpE, 75) fracGpE, apmiGene, apmiEnh, 76) fracGpE, apmiGene, apmiGmE, 77) fracGpE, apmiGene, apmiGpE, 78) fracGpE, apmiEnh, apmiGmE, 79) fracGpE, apmiEnh, apmiGpE, 80) fracGpE, apmiGmE, apmiGpE, 81) apmiGene, apmiEnh, apmiGmE, 82) apmiGene, apmiEnh, apmiGpE, 83) apmiGene, apmiGmE, apmiGpE, and 84) apmiEnh, apmiGmE, apmiGpE.

In various embodiments, machine models disclosed herein include 4 engineered features selected from APMI, fracEnh, fracGene, fracGmE, fracGpE, apmiGene, apmiEnh, apmiGmE, or apmiGpE. In various embodiments, machine models disclosed herein include 4 engineered features, including combinations such as 1) APMI, fracEnh, fracGene, fracGmE, 2) APMI, fracEnh, fracGene, fracGmE, 3) APMI, fracEnh, fracGene, apmiGene, 4) APMI, fracEnh, fracGene, apmiEnh, 5) APMI, fracEnh, fracGene, apmiGmE, 6) APMI, fracEnh, fracGene, apmiGpE, 7) APMI, fracEnh, fracGmE, fracGmE, 8) APMI, fracEnh, fracGmE, apmiGene, 9) APMI, fracEnh, fracGmE, apmiEnh, 10) APMI, fracEnh, fracGmE, apmiGmE, 11) APMI, fracEnh, fracGmE, apmiGpE, 12) APMI, fracEnh, fracGmE, apmiGene, 13) APMI, fracEnh, fracGmE, apmiEnh, 14) APMI, fracEnh, fracGmE, apmiGmE, 15) APMI, fracEnh, fracGmE, apmiGpE, 16) APMI, fracEnh, apmiGene, apmiEnh, 17) APMI, fracEnh, apmiGene, apmiGmE, 18) APMI, fracEnh, apmiGene, apmiGpE, 19) APMI, fracEnh, apmiEnh, apmiGmE, 20) APMI, fracEnh, apmiEnh, apmiGpE, 21) APMI, fracEnh, apmiGmE, apmiGpE, 22) APMI, fracGene, fracGmE, fracGmE, 23) APMI, fracGene, fracGmE, apmiGene, 24) APMI, fracGene, fracGmE, apmiEnh, 25) APMI, fracGene, fracGmE, apmiGmE, 26) APMI, fracGene, fracGmE, apmiGpE, 27) APMI, fracGene, fracGmE, apmiGene, 28) APMI, fracGene, fracGmE, apmiEnh, 29) APMI, fracGene, fracGmE, apmiGmE, 30) APMI, fracGene, fracGmE, apmiGpE, 31) APMI, fracGene, apmiGene, apmiEnh, 32) APMI, fracGene, apmiGene, apmiGmE, 33) APMI, fracGene, apmiGene, apmiGpE, 34) APMI, fracGene, apmiEnh, apmiGmE, 35) APMI, fracGene, apmiEnh, apmiGpE, 36) APMI, fracGene, apmiGmE, apmiGpE, 37) APMI, fracGmE, fracGmE, apmiGene, 38) APMI, fracGmE, fracGmE, apmiEnh, 39) APMI, fracGmE, fracGmE, apmiGmE, 40) APMI, fracGmE, fracGmE, apmiGpE, 41) APMI, fracGmE, apmiGene, apmiEnh, 42) APMI, fracGmE, apmiGene, apmiGmE, 43) APMI, fracGmE, apmiGene, apmiGpE, 44) APMI, fracGmE, apmiEnh, apmiGmE, 45) APMI, fracGmE, apmiEnh, apmiGpE, 46) APMI, fracGmE, apmiGmE, apmiGpE, 47) APMI, fracGmE, apmiGene, apmiEnh, 48) APMI, fracGmE, apmiGene, apmiGmE, 49) APMI, fracGmE, apmiGene, apmiGpE, 50) APMI, fracGmE, apmiEnh, apmiGmE, 51) APMI, fracGmE, apmiEnh, apmiGpE, 52) APMI, fracGmE, apmiGmE, apmiGpE, 53) APMI, apmiGene, apmiEnh, apmiGmE, 54) APMI, apmiGene, apmiEnh, apmiGpE, 55) APMI, apmiGene, apmiGmE, apmiGpE, 56) APMI, apmiEnh, apmiGmE, apmiGpE, 57) fracEnh, fracGene, fracGmE, fracGmE, 58) fracEnh, fracGene, fracGmE, apmiGene, 59) fracEnh, fracGene, fracGmE, apmiEnh, 60) fracEnh, fracGene, fracGmE, apmiGmE, 61) fracEnh, fracGene, fracGmE, apmiGpE, 62) fracEnh, fracGene, fracGmE, apmiGene, 63) fracEnh, fracGene, fracGmE, apmiEnh, 64) fracEnh, fracGene, fracGmE, apmiGmE, 65) fracEnh, fracGene, fracGmE, apmiGpE, 66) fracEnh, fracGene, apmiGene, apmiEnh, 67) fracEnh, fracGene, apmiGene, apmiGmE, 68) fracEnh, fracGene, apmiGene, apmiGpE, 69) fracEnh, fracGene, apmiEnh, apmiGmE, 70) fracEnh, fracGene, apmiEnh, apmiGpE, 71) fracEnh, fracGene, apmiGmE, apmiGpE, 72) fracEnh, fracGmE, fracGmE, apmiGene, 73) fracEnh, fracGmE, fracGmE, apmiEnh, 74) fracEnh, fracGmE, fracGmE, apmiGmE, 75) fracEnh, fracGmE, fracGmE, apmiGpE, 76) fracEnh, fracGmE, apmiGene, apmiEnh, 77) fracEnh, fracGmE, apmiGene, apmiGmE, 78) fracEnh, fracGmE, apmiGene, apmiGpE, 79) fracEnh, fracGmE, apmiEnh, apmiGmE, 80) fracEnh, fracGmE, apmiEnh, apmiGpE, 81) fracEnh, fracGmE, apmiGmE, apmiGpE, 82) fracEnh, fracGmE, apmiGene, apmiEnh, 83) fracEnh, fracGmE, apmiGene, apmiGmE, 84) fracEnh, fracGmE, apmiGene, apmiGpE, 85) fracEnh, fracGmE, apmiEnh, apmiGmE, 86) fracEnh, fracGmE, apmiEnh, apmiGpE, 87) fracEnh, fracGmE, apmiGmE, apmiGpE, 88) fracEnh, apmiGene, apmiEnh, apmiGmE, 89) fracEnh, apmiGene, apmiEnh, apmiGpE, 90) fracEnh, apmiGene, apmiGmE, apmiGpE, 91) fracEnh, apmiEnh, apmiGmE, apmiGpE, 92) fracGene, fracGmE, fracGmE, apmiGene, 93) fracGene, fracGmE, fracGmE, apmiEnh, 94) fracGene, fracGmE, fracGmE, apmiGmE, 95) fracGene, fracGmE, fracGmE, apmiGpE, 96) fracGene, fracGmE, apmiGene, apmiEnh, 97) fracGene, fracGmE, apmiGene, apmiGmE, 98) fracGene, fracGmE, apmiGene, apmiGpE, 99) fracGene, fracGmE, apmiEnh, apmiGmE, 100) fracGene, fracGmE, apmiEnh, apmiGpE, 101) fracGene, fracGmE, apmiGmE, apmiGpE, 102) fracGene, fracGmE, apmiGene, apmiEnh, 103) fracGene, fracGmE, apmiGene, apmiGmE, 104) fracGene, fracGmE, apmiGene, apmiGpE, 105) fracGene, fracGmE, apmiEnh, apmiGmE, 106) fracGene, fracGmE, apmiEnh, apmiGpE, 107) fracGene, fracGmE, apmiGmE, apmiGpE, 108) fracGene, apmiGene, apmiEnh, apmiGmE, 109) fracGene, apmiGene, apmiEnh, apmiGpE, 110) fracGene, apmiGene, apmiGmE, apmiGpE, 111) fracGene, apmiEnh, apmiGmE, apmiGpE, 112) fracGmE, fracGmE, apmiGene, apmiEnh, 113) fracGmE, fracGmE, apmiGene, apmiGmE, 114) fracGmE, fracGmE, apmiGene, apmiGpE, 115) fracGmE, fracGmE, apmiEnh, apmiGmE, 116) fracGmE, fracGmE, apmiEnh, apmiGpE, 117) fracGmE, fracGmE, apmiGmE, apmiGpE, 118) fracGmE, apmiGene, apmiEnh, apmiGmE, 119) fracGmE, apmiGene, apmiEnh, apmiGpE, 120) fracGmE, apmiGene, apmiGmE, apmiGpE, 121) fracGmE, apmiEnh, apmiGmE, apmiGpE, 122) fracGmE, apmiGene, apmiEnh, apmiGmE, 123) fracGmE, apmiGene, apmiEnh, apmiGpE, 124) fracGmE, apmiGene, apmiGmE, apmiGpE, 125) fracGmE, apmiEnh, apmiGmE, apmiGpE, and 126) apmiGene, apmiEnh, apmiGmE, apmiGpE.

In various embodiments, machine models disclosed herein include 5 engineered features selected from APMI, fracEnh, fracGene, fracGmE, fracGpE, apmiGene, apmiEnh, apmiGmE, or apmiGpE. In various embodiments, machine models disclosed herein include 5 engineered features, including combinations such as 1) APMI, fracEnh, fracGene, fracGmE, fracGpE, 2) APMI, fracEnh, fracGene, fracGmE, apmiGene, 3) APMI, fracEnh, fracGene, fracGmE, apmiEnh, 4) APMI, fracEnh, fracGene, fracGmE, apmiGmE, 5) APMI, fracEnh, fracGene, fracGmE, apmiGpE, 6) APMI, fracEnh, fracGene, fracGpE, apmiGene, 7) APMI, fracEnh, fracGene, fracGpE, apmiEnh, 8) APMI, fracEnh, fracGene, fracGpE, apmiGmE, 9) APMI, fracEnh, fracGene, fracGpE, apmiGpE, 10) APMI, fracEnh, fracGene, apmiGene, apmiEnh, 11) APMI, fracEnh, fracGene, apmiGene, apmiGmE, 12) APMI, fracEnh, fracGene, apmiGene, apmiGpE, 13) APMI, fracEnh, fracGene, apmiEnh, apmiGmE, 14) APMI, fracEnh, fracGene, apmiEnh, apmiGpE, 15) APMI, fracEnh, fracGene, apmiGmE, apmiGpE, 16) APMI, fracEnh, fracGmE, fracGpE, apmiGene, 17) APMI, fracEnh, fracGmE, fracGpE, apmiEnh, 18) APMI, fracEnh, fracGmE, fracGpE, apmiGmE, 19) APMI, fracEnh, fracGmE, fracGpE, apmiGpE, 20) APMI, fracEnh, fracGmE, apmiGene, apmiEnh, 21) APMI, fracEnh, fracGmE, apmiGene, apmiGmE, 22) APMI, fracEnh, fracGmE, apmiGene, apmiGpE, 23) APMI, fracEnh, fracGmE, apmiEnh, apmiGmE, 24) APMI, fracEnh, fracGmE, apmiEnh, apmiGpE, 25) APMI, fracEnh, fracGmE, apmiGmE, apmiGpE, 26) APMI, fracEnh, fracGpE, apmiGene, apmiEnh, 27) APMI, fracEnh, fracGpE, apmiGene, apmiGmE, 28) APMI, fracEnh, fracGpE, apmiGene, apmiGpE, 29) APMI, fracEnh, fracGpE, apmiEnh, apmiGmE, 30) APMI, fracEnh, fracGpE, apmiEnh, apmiGpE, 31) APMI, fracEnh, fracGpE, apmiGmE, apmiGpE, 32) APMI, fracEnh, apmiGene, apmiEnh, apmiGmE, 33) APMI, fracEnh, apmiGene, apmiEnh, apmiGpE, 34) APMI, fracEnh, apmiGene, apmiGmE, apmiGpE, 35) APMI, fracEnh, apmiEnh, apmiGmE, apmiGpE, 36) APMI, fracGene, fracGmE, fracGpE, apmiGene, 37) APMI, fracGene, fracGmE, fracGpE, apmiEnh, 38) APMI, fracGene, fracGmE, fracGpE, apmiGmE, 39) APMI, fracGene, fracGmE, fracGpE, apmiGpE, 40) APMI, fracGene, fracGmE, apmiGene, apmiEnh, 41) APMI, fracGene, fracGmE, apmiGene, apmiGmE, 42) APMI, fracGene, fracGmE, apmiGene, apmiGpE, 43) APMI, fracGene, fracGmE, apmiEnh, apmiGmE, 44) APMI, fracGene, fracGmE, apmiEnh, apmiGpE, 45) APMI, fracGene, fracGmE, apmiGmE, apmiGpE, 46) APMI, fracGene, fracGpE, apmiGene, apmiEnh, 47) APMI, fracGene, fracGpE, apmiGene, apmiGmE, 48) APMI, fracGene, fracGpE, apmiGene, apmiGpE, 49) APMI, fracGene, fracGpE, apmiEnh, apmiGmE, 50) APMI, fracGene, fracGpE, apmiEnh, apmiGpE, 51) APMI, fracGene, fracGpE, apmiGmE, apmiGpE, 52) APMI, fracGene, apmiGene, apmiEnh, apmiGmE, 53) APMI, fracGene, apmiGene, apmiEnh, apmiGpE, 54) APMI, fracGene, apmiGene, apmiGmE, apmiGpE, 55) APMI, fracGene, apmiEnh, apmiGmE, apmiGpE, 56) APMI, fracGmE, fracGpE, apmiGene, apmiEnh, 57) APMI, fracGmE, fracGpE, apmiGene, apmiGmE, 58) APMI, fracGmE, fracGpE, apmiGene, apmiGpE, 59) APMI, fracGmE, fracGpE, apmiEnh, apmiGmE, 60) APMI, fracGmE, fracGpE, apmiEnh, apmiGpE, 61) APMI, fracGmE, fracGpE, apmiGmE, apmiGpE, 62) APMI, fracGmE, apmiGene, apmiEnh, apmiGmE, 63) APMI, fracGmE, apmiGene, apmiEnh, apmiGpE, 64) APMI, fracGmE, apmiGene, apmiGmE, apmiGpE, 65) APMI, fracGmE, apmiEnh, apmiGmE, apmiGpE, 66) APMI, fracGpE, apmiGene, apmiEnh, apmiGmE, 67) APMI, fracGpE, apmiGene, apmiEnh, apmiGpE, 68) APMI, fracGpE, apmiGene, apmiGmE, apmiGpE, 69) APMI, fracGpE, apmiEnh, apmiGmE, apmiGpE, 70) APMI, apmiGene, apmiEnh, apmiGmE, apmiGpE, 71) fracEnh, fracGene, fracGmE, fracGpE, apmiGene, 72) fracEnh, fracGene, fracGmE, fracGpE, apmiEnh, 73) fracEnh, fracGene, fracGmE, fracGpE, apmiGmE, 74) fracEnh, fracGene, fracGmE, fracGpE, apmiGpE, 75) fracEnh, fracGene, fracGmE, apmiGene, apmiEnh, 76) fracEnh, fracGene, fracGmE, apmiGene, apmiGmE, 77) fracEnh, fracGene, fracGmE, apmiGene, apmiGpE, 78) fracEnh, fracGene, fracGmE, apmiEnh, apmiGmE, 79) fracEnh, fracGene, fracGmE, apmiEnh, apmiGpE, 80) fracEnh, fracGene, fracGmE, apmiGmE, apmiGpE, 81) fracEnh, fracGene, fracGpE, apmiGene, apmiEnh, 82) fracEnh, fracGene, fracGpE, apmiGene, apmiGmE, 83) fracEnh, fracGene, fracGpE, apmiGene, apmiGpE, 84) fracEnh, fracGene, fracGpE, apmiEnh, apmiGmE, 85) fracEnh, fracGene, fracGpE, apmiEnh, apmiGpE, 86) fracEnh, fracGene, fracGpE, apmiGmE, apmiGpE, 87) fracEnh, fracGene, apmiGene, apmiEnh, apmiGmE, 88) fracEnh, fracGene, apmiGene, apmiEnh, apmiGpE, 89) fracEnh, fracGene, apmiGene, apmiGmE, apmiGpE, 90) fracEnh, fracGene, apmiEnh, apmiGmE, apmiGpE, 91) fracEnh, fracGmE, fracGpE, apmiGene, apmiEnh, 92) fracEnh, fracGmE, fracGpE, apmiGene, apmiGmE, 93) fracEnh, fracGmE, fracGpE, apmiGene, apmiGpE, 94) fracEnh, fracGmE, fracGpE, apmiEnh, apmiGmE, 95) fracEnh, fracGmE, fracGpE, apmiEnh, apmiGpE, 96) fracEnh, fracGmE, fracGpE, apmiGmE, apmiGpE, 97) fracEnh, fracGmE, apmiGene, apmiEnh, apmiGmE, 98) fracEnh, fracGmE, apmiGene, apmiEnh, apmiGpE, 99) fracEnh, fracGmE, apmiGene, apmiGmE, apmiGpE, 100) fracEnh, fracGmE, apmiEnh, apmiGmE, apmiGpE, 101) fracEnh, fracGpE, apmiGene, apmiEnh, apmiGmE, 102) fracEnh, fracGpE, apmiGene, apmiEnh, apmiGpE, 103) fracEnh, fracGpE, apmiGene, apmiGmE, apmiGpE, 104) fracEnh, fracGpE, apmiEnh, apmiGmE, apmiGpE, 105) fracEnh, apmiGene, apmiEnh, apmiGmE, apmiGpE, 106) fracGene, fracGmE, fracGpE, apmiGene, apmiEnh, 107) fracGene, fracGmE, fracGpE, apmiGene, apmiGmE, 108) fracGene, fracGmE, fracGpE, apmiGene, apmiGpE, 109) fracGene, fracGmE, fracGpE, apmiEnh, apmiGmE, 110) fracGene, fracGmE, fracGpE, apmiEnh, apmiGpE, 111) fracGene, fracGmE, fracGpE, apmiGmE, apmiGpE, 112) fracGene, fracGmE, apmiGene, apmiEnh, apmiGmE, 113) fracGene, fracGmE, apmiGene, apmiEnh, apmiGpE, 114) fracGene, fracGmE, apmiGene, apmiGmE, apmiGpE, 115) fracGene, fracGmE, apmiEnh, apmiGmE, apmiGpE, 116) fracGene, fracGpE, apmiGene, apmiEnh, apmiGmE, 117) fracGene, fracGpE, apmiGene, apmiEnh, apmiGpE, 118) fracGene, fracGpE, apmiGene, apmiGmE, apmiGpE, 119) fracGene, fracGpE, apmiEnh, apmiGmE, apmiGpE, 120) fracGene, apmiGene, apmiEnh, apmiGmE, apmiGpE, 121) fracGmE, fracGpE, apmiGene, apmiEnh, apmiGmE, 122) fracGmE, fracGpE, apmiGene, apmiEnh, apmiGpE, 123) fracGmE, fracGpE, apmiGene, apmiGmE, apmiGpE, 124) fracGmE, fracGpE, apmiEnh, apmiGmE, apmiGpE, 125) fracGmE, apmiGene, apmiEnh, apmiGmE, apmiGpE, and 126) fracGpE, apmiGene, apmiEnh, apmiGmE, apmiGpE.

In various embodiments, machine models disclosed herein include 6 engineered features selected from APMI, fracEnh, fracGene, fracGmE, fracGpE, apmiGene, apmiEnh, apmiGmE, or apmiGpE. In various embodiments, machine models disclosed herein include 6 engineered features, including combinations such as: 1) APMI, fracEnh, fracGene, fracGmE, fracGpE, apmiGene, 2) APMI, fracEnh, fracGene, fracGmE, fracGpE, apmiEnh, 3) APMI, fracEnh, fracGene, fracGmE, fracGpE, apmiGmE, 4) APMI, fracEnh, fracGene, fracGmE, fracGpE, apmiGpE, 5) APMI, fracEnh, fracGene, fracGmE, apmiGene, apmiEnh, 6) APMI, fracEnh, fracGene, fracGmE, apmiGene, apmiGmE, 7) APMI, fracEnh, fracGene, fracGmE, apmiGene, apmiGpE, 8) APMI, fracEnh, fracGene, fracGmE, apmiEnh, apmiGmE, 9) APMI, fracEnh, fracGene, fracGmE, apmiEnh, apmiGpE, 10) APMI, fracEnh, fracGene, fracGmE, apmiGmE, apmiGpE, 11) APMI, fracEnh, fracGene, fracGpE, apmiGene, apmiEnh, 12) APMI, fracEnh, fracGene, fracGpE, apmiGene, apmiGmE, 13) APMI, fracEnh, fracGene, fracGpE, apmiGene, apmiGpE, 14) APMI, fracEnh, fracGene, fracGpE, apmiEnh, apmiGmE, 15) APMI, fracEnh, fracGene, fracGpE, apmiEnh, apmiGpE, 16) APMI, fracEnh, fracGene, fracGpE, apmiGmE, apmiGpE, 17) APMI, fracEnh, fracGene, apmiGene, apmiEnh, apmiGmE, 18) APMI, fracEnh, fracGene, apmiGene, apmiEnh, apmiGpE, 19) APMI, fracEnh, fracGene, apmiGene, apmiGmE, apmiGpE, 20) APMI, fracEnh, fracGene, apmiEnh, apmiGmE, apmiGpE, 21) APMI, fracEnh, fracGmE, fracGpE, apmiGene, apmiEnh, 22) APMI, fracEnh, fracGmE, fracGpE, apmiGene, apmiGmE, 23) APMI, fracEnh, fracGmE, fracGpE, apmiGene, apmiGpE, 24) APMI, fracEnh, fracGmE, fracGpE, apmiEnh, apmiGmE, 25) APMI, fracEnh, fracGmE, fracGpE, apmiEnh, apmiGpE, 26) APMI, fracEnh, fracGmE, fracGpE, apmiGmE, apmiGpE, 27) APMI, fracEnh, fracGmE, apmiGene, apmiEnh, apmiGmE, 28) APMI, fracEnh, fracGmE, apmiGene, apmiEnh, apmiGpE, 29) APMI, fracEnh, fracGmE, apmiGene, apmiGmE, apmiGpE, 30) APMI, fracEnh, fracGmE, apmiEnh, apmiGmE, apmiGpE, 31) APMI, fracEnh, fracGpE, apmiGene, apmiEnh, apmiGmE, 32) APMI, fracEnh, fracGpE, apmiGene, apmiEnh, apmiGpE, 33) APMI, fracEnh, fracGpE, apmiGene, apmiGmE, apmiGpE, 34) APMI, fracEnh, fracGpE, apmiEnh, apmiGmE, apmiGpE, 35) APMI, fracEnh, apmiGene, apmiEnh, apmiGmE, apmiGpE, 36) APMI, fracGene, fracGmE, fracGpE, apmiGene, apmiEnh, 37) APMI, fracGene, fracGmE, fracGpE, apmiGene, apmiGmE, 38) APMI, fracGene, fracGmE, fracGpE, apmiGene, apmiGpE, 39) APMI, fracGene, fracGmE, fracGpE, apmiEnh, apmiGmE, 40) APMI, fracGene, fracGmE, fracGpE, apmiEnh, apmiGpE, 41) APMI, fracGene, fracGmE, fracGpE, apmiGmE, apmiGpE, 42) APMI, fracGene, fracGmE, apmiGene, apmiEnh, apmiGmE, 43) APMI, fracGene, fracGmE, apmiGene, apmiEnh, apmiGpE, 44) APMI, fracGene, fracGmE, apmiGene, apmiGmE, apmiGpE, 45) APMI, fracGene, fracGmE, apmiEnh, apmiGmE, apmiGpE, 46) APMI, fracGene, fracGpE, apmiGene, apmiEnh, apmiGmE, 47) APMI, fracGene, fracGpE, apmiGene, apmiEnh, apmiGpE, 48) APMI, fracGene, fracGpE, apmiGene, apmiGmE, apmiGpE, 49) APMI, fracGene, fracGpE, apmiEnh, apmiGmE, apmiGpE, 50) APMI, fracGene, apmiGene, apmiEnh, apmiGmE, apmiGpE, 51) APMI, fracGmE, fracGpE, apmiGene, apmiEnh, apmiGmE, 52) APMI, fracGmE, fracGpE, apmiGene, apmiEnh, apmiGpE, 53) APMI, fracGmE, fracGpE, apmiGene, apmiGmE, apmiGpE, 54) APMI, fracGmE, fracGpE, apmiEnh, apmiGmE, apmiGpE, 55) APMI, fracGmE, apmiGene, apmiEnh, apmiGmE, apmiGpE, 56) APMI, fracGpE, apmiGene, apmiEnh, apmiGmE, apmiGpE, 57) fracEnh, fracGene, fracGmE, fracGpE, apmiGene, apmiEnh, 58) fracEnh, fracGene, fracGmE, fracGpE, apmiGene, apmiGmE, 59) fracEnh, fracGene, fracGmE, fracGpE, apmiGene, apmiGpE, 60) fracEnh, fracGene, fracGmE, fracGpE, apmiEnh, apmiGmE, 61) fracEnh, fracGene, fracGmE, fracGpE, apmiEnh, apmiGpE, 62) fracEnh, fracGene, fracGmE, fracGpE, apmiGmE, apmiGpE, 63) fracEnh, fracGene, fracGmE, apmiGene, apmiEnh, apmiGmE, 64) fracEnh, fracGene, fracGmE, apmiGene, apmiEnh, apmiGpE, 65) fracEnh, fracGene, fracGmE, apmiGene, apmiGmE, apmiGpE, 66) fracEnh, fracGene, fracGmE, apmiEnh, apmiGmE, apmiGpE, 67) fracEnh, fracGene, fracGpE, apmiGene, apmiEnh, apmiGmE, 68) fracEnh, fracGene, fracGpE, apmiGene, apmiEnh, apmiGpE, 69) fracEnh, fracGene, fracGpE, apmiGene, apmiGmE, apmiGpE, 70) fracEnh, fracGene, fracGpE, apmiEnh, apmiGmE, apmiGpE, 71) fracEnh, fracGene, apmiGene, apmiEnh, apmiGmE, apmiGpE, 72) fracEnh, fracGmE, fracGpE, apmiGene, apmiEnh, apmiGmE, 73) fracEnh, fracGmE, fracGpE, apmiGene, apmiEnh, apmiGpE, 74) fracEnh, fracGmE, fracGpE, apmiGene, apmiGmE, apmiGpE, 75) fracEnh, fracGmE, fracGpE, apmiEnh, apmiGmE, apmiGpE, 76) fracEnh, fracGmE, apmiGene, apmiEnh, apmiGmE, apmiGpE, 77) fracEnh, fracGpE, apmiGene, apmiEnh, apmiGmE, apmiGpE, 78) fracGene, fracGmE, fracGpE, apmiGene, apmiEnh, apmiGmE, 79) fracGene, fracGmE, fracGpE, apmiGene, apmiEnh, apmiGpE, 80) fracGene, fracGmE, fracGpE, apmiGene, apmiGmE, apmiGpE, 81) fracGene, fracGmE, fracGpE, apmiEnh, apmiGmE, apmiGpE, 82) fracGene, fracGmE, apmiGene, apmiEnh, apmiGmE, apmiGpE, 83) fracGene, fracGpE, apmiGene, apmiEnh, apmiGmE, apmiGpE, and 84) fracGmE, fracGpE, apmiGene, apmiEnh, apmiGmE, apmiGpE,

In various embodiments, machine models disclosed herein include 7 engineered features including combinations such as: 1) APMI, fracEnh, fracGene, fracGmE, fracGpE, apmiGene, apmiEnh, 2) APMI, fracEnh, fracGene, fracGmE, fracGpE, apmiGene, apmiGmE, 3) APMI, fracEnh, fracGene, fracGmE, fracGpE, apmiGene, apmiGpE, 4) APMI, fracEnh, fracGene, fracGmE, fracGpE, apmiEnh, apmiGmE, 5) APMI, fracEnh, fracGene, fracGmE, fracGpE, apmiEnh, apmiGpE, 6) APMI, fracEnh, fracGene, fracGmE, fracGpE, apmiGmE, apmiGpE, 7) APMI, fracEnh, fracGene, fracGmE, apmiGene, apmiEnh, apmiGmE, 8) APMI, fracEnh, fracGene, fracGmE, fracGpE, apmiEnh, apmiGpE, 9) APMI, fracEnh, fracGene, fracGmE, fracGpE, apmiGmE, apmiGpE, 10) APMI, fracEnh, fracGene, fracGmE, apmiEnh, apmiGmE, apmiGpE, 11) APMI, fracEnh, fracGene, fracGpE, fracGpE, apmiEnh, apmiGmE, 12) APMI, fracEnh, fracGene, fracGpE, fracGpE, apmiEnh, apmiGpE, 13) APMI, fracEnh, fracGene, fracGpE, fracGpE, apmiGmE, apmiGpE, 14) APMI, fracEnh, fracGene, fracGpE, apmiEnh, apmiGmE, apmiGpE, 15) APMI, fracEnh, fracGene, fracGpE, apmiEnh, apmiGmE, apmiGpE, 16) APMI, fracEnh, fracGmE, fracGpE, fracGpE, apmiEnh, apmiGmE, 17) APMI, fracEnh, fracGmE, fracGpE, fracGpE, apmiEnh, apmiGpE, 18) APMI, fracEnh, fracGmE, fracGpE, fracGpE, apmiGmE, apmiGpE, 19) APMI, fracEnh, fracGmE, fracGpE, apmiEnh, apmiGmE, apmiGpE, 20) APMI, fracEnh, fracGmE, fracGpE, apmiEnh, apmiGmE, apmiGpE, 21) APMI, fracEnh, fracGpE, fracGpE, apmiEnh, apmiGmE, apmiGpE, 22) APMI, fracGene, fracGmE, fracGpE, fracGpE, apmiEnh, apmiGmE, 23) APMI, fracGene, fracGmE, fracGpE, fracGpE, apmiEnh, apmiGpE, 24) APMI, fracGene, fracGmE, fracGpE, fracGpE, apmiGmE, apmiGpE, 25) APMI, fracGene, fracGmE, fracGpE, apmiEnh, apmiGmE, apmiGpE, 26) APMI, fracGene, fracGmE, fracGpE, apmiEnh, apmiGmE, apmiGpE, 27) APMI, fracGene, fracGpE, fracGpE, apmiEnh, apmiGmE, apmiGpE, 28) APMI, fracGmE, fracGpE, fracGpE, apmiEnh, apmiGmE, apmiGpE, 29) fracEnh, fracGene, fracGmE, fracGpE, fracGpE, apmiEnh, apmiGmE, 30) fracEnh, fracGene, fracGmE, fracGpE, fracGpE, apmiEnh, apmiGpE, 31) fracEnh, fracGene, fracGmE, fracGpE, fracGpE, apmiGmE, apmiGpE, 32) fracEnh, fracGene, fracGmE, fracGpE, apmiEnh, apmiGmE, apmiGpE, 33) fracEnh, fracGene, fracGmE, fracGpE, apmiEnh, apmiGmE, apmiGpE, 34) fracEnh, fracGene, fracGpE, fracGpE, apmiEnh, apmiGmE, apmiGpE, 35) fracEnh, fracGmE, fracGpE, fracGpE, apmiEnh, apmiGmE, apmiGpE, and 36) fracGene, fracGmE, fracGpE, fracGpE, apmiEnh, apmiGmE, apmiGpE,

In various embodiments, machine models disclosed herein include 8 engineered features, including 1) fracEnh, fracGene, fracGmE, fracGpE, apmiGene, apmiEnh, apmiGmE, and apmiGpE, 2) APMI, fracGene, fracGmE, fracGpE, apmiGene, apmiEnh, apmiGmE, and apmiGpE, 3) APMI, fracEnh, fracGmE, fracGpE, apmiGene, apmiEnh, apmiGmE, and apmiGpE, 4) APMI, fracEnh, fracGene, fracGpE, apmiGene, apmiEnh, apmiGmE, and apmiGpE, 5) APMI, fracEnh, fracGene, fracGmE, apmiGene, apmiEnh, apmiGmE, and apmiGpE, 6) APMI, fracEnh, fracGene, fracGmE, fracGpE, apmiEnh, apmiGmE, and apmiGpE, 7) APMI, fracEnh, fracGene, fracGmE, fracGpE, apmiGene, apmiEnh, and apmiGpE, or 8) APMI, fracEnh, fracGene, fracGmE, fracGpE, apmiGene, apmiEnh, and apmiGmE.

In various embodiments, machine models disclosed herein include 9 engineered features, including each of APMI, fracEnh, fracGene, fracGmE, fracGpE, apmiGene, apmiEnh, apmiGmE, or apmiGpE.

### Example Machine Learning Model for Predicting Functional Enhancer Promoter Pairs

Disclosed herein is the training and deployment of machine learning models for predicting whether an enhancer-promoter pair is a functional or non-functional enhancer-promoter pair. In various embodiments, a machine learning model is any one of a regression model (e.g., linear regression, polynomial regression, or generalized linear model(GLM)), decision tree, random forest, boosting, gradient boosting, support vector machine, logistic regression, Naive Bayes model, K-Nearest Neighbors, or neural network (e.g., feed-forward networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks). In particular embodiments, the machine learning model is a random forest model.

The machine learning model can be trained using a machine learning implemented method, such as any one of a linear regression algorithm, logistic regression algorithm, decision tree algorithm, support vector machine classification, Naive Bayes classification, K-Nearest Neighbors classification, random forest algorithm, deep learning algorithm, gradient boosting algorithm, and dimensionality reduction techniques such as manifold learning, principal component analysis, factor analysis, autoencoder regularization, and independent component analysis, or combinations thereof. In various embodiments, the machine learning model has one or more parameters, such as hyperparameters or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of neural network, support vectors in a support vector machine, and coefficients in a regression model. The model parameters of the machine learning model are trained (e.g., adjusted) using the training data to improve the predictive power of the machine learning model.

Different features of the machine learning model may differently influence the prediction outputted by the machine learning model. For example, different features of the machine learning model may have different feature importance values. Thus, features with higher feature importance values may more heavily influence the prediction outputted by the machine learning model whereas features with lower feature importance values may less heavily influence the prediction outputted by the machine learning model.

As disclosed herein, a machine learning model may include a first set of features and a second set of features engineered from a subset of the first set of features. In various embodiments, at least one feature of the second set has a higher feature importance value in comparison to at least one feature of the first set. In various embodiments, at least three features of the second set have a higher feature importance value in comparison to at least three features of the first set. In various embodiments, at least five features of the second set have a higher feature importance value in comparison to at least five features of the first set. In various embodiments, each feature of the second set has a higher feature importance value in comparison to each feature of the first set.

The performance of a machine learning model is generally characterized according to one or more metrics. Example metrics include an accuracy metric, precision metric, an area under a precision recall curve (AUPR) metric, an area under a receiver operating characteristic curve (AUROC) metric, a positive predictive value (PPV), or a negative predictive value (NPV).

In various embodiments, machine learning models disclosed herein achieve an area under a precision recall curve (AUPR) metric of at least 0.55. In various embodiments, machine learning models disclosed herein achieve an area under a precision recall curve (AUPR) metric of at least 0.60. In various embodiments, machine learning models disclosed herein achieve an area under a precision recall curve (AUPR) metric of at least 0.65, at least 0.70, at least 0.75, at least 0.80, at least 0.85, at least 0.90, at least 0.91, at least 0.92, at least 0.93, at least 0.94, at least 0.95, at least 0.96, at least 0.97, at least 0.98, or at least 0.99. In various embodiments, machine learning models disclosed herein achieve an area under a receiver operative curve (AUROC) metric of at least 0.90. In various embodiments, machine learning models disclosed herein achieve an area under a receiver operative curve (AUROC) metric of at least 0.91. In various embodiments, machine learning models disclosed herein achieve an area under a receiver operative curve (AUROC) metric of at least 0.92, at least 0.93, at least 0.94, at least 0.95, at least 0.96, at least 0.97, at least 0.98, or at least 0.99.

### Computing Device

The methods described above, including the methods of predicting functional enhancer-promoter pairs, are, in some embodiments, performed on a computing device. Examples of a computing device can include a personal computer, desktop computer laptop, server computer, a computing node within a cluster, message processors, hand-held devices, multi-processor systems, microprocessor-based or programmable consumer electronics, network PCs, minicomputers, mainframe computers, mobile telephones, PDAs, tablets, pagers, routers, switches, and the like.

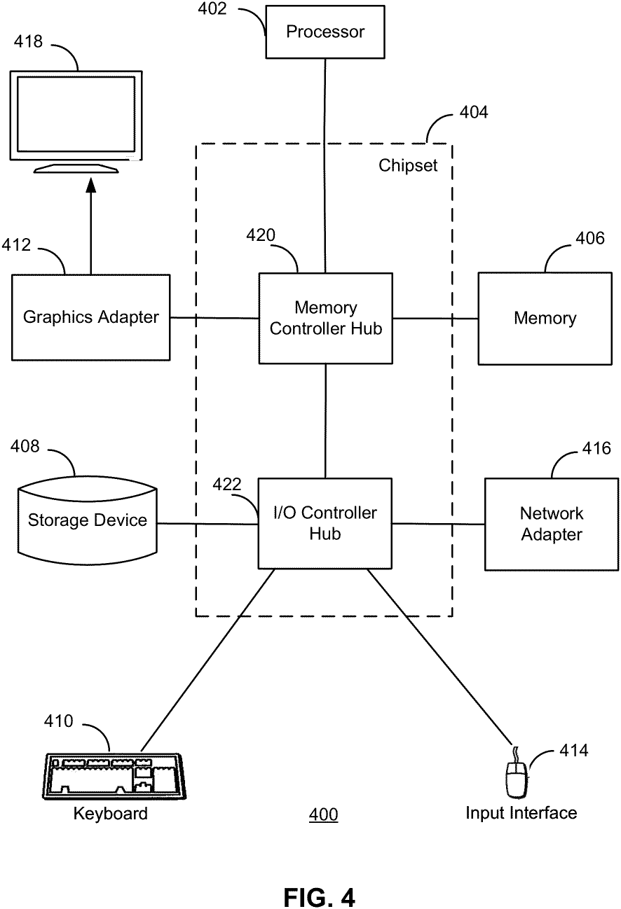

FIG. 4 illustrates an example computing device 400 for implementing system and methods described in FIGS. 1, 1A, 2B, 3A, or 3B. In some embodiments, the computing device 400 includes at least one processor 402 coupled to a chipset 404. The chipset 404 includes a memory controller hub 420 and an input/output (I/O) controller hub 422. A memory 406 and a graphics adapter 412 are coupled to the memory controller hub 420, and a display 418 is coupled to the graphics adapter 412. In some embodiments, a computing device 400 can include a processor 402 for executing instructions stored on a memory 406. A storage device 408, an input interface 414, and network adapter 416 are coupled to the I/O controller hub 422. Other embodiments of the computing device 400 have different architectures.

The storage device 408 is a non-transitory computer-readable storage medium such as a hard drive, compact disk read-only memory (CD-ROM), DVD, or a solid-state memory device. The memory 406 holds instructions and data used by the processor 402. The input interface 414 is a touch-screen interface, a mouse, track ball, or other type of input interface, a keyboard 410, or some combination thereof, and is used to input data into the computing device 400. In some embodiments, the computing device 400 may be configured to receive input (e.g., commands) from the input interface 414 via gestures from the user. The graphics adapter 412 displays images and other information on the display 418. For example, the display 418 can show an indication of a predicted functional enhancer-promoter or a predicted non-functional enhancer-promoter. The network adapter 416 couples the computing device 400 to one or more computer networks.

The computing device 400 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 408, loaded into the memory 406, and executed by the processor 402.

The types of computing devices 400 can vary from the embodiments described herein. For example, the computing device 400 can lack some of the components described above, such as graphics adapters 412, input interface 414, and displays 418.

The methods disclosed herein for predicting functional or non-functional enhancer-promoter pairs can be implemented in hardware or software, or a combination of both. In one embodiment, a non-transitory machine-readable storage medium, such as one described above, is provided, the medium comprising a data storage material encoded with machine readable data which, when using a machine programmed with instructions for using said data, is capable of displaying any of the datasets and execution and results of a machine learning model of this invention. Embodiments of the methods described above can be implemented in computer programs executing on programmable computers, comprising a processor, a data storage system (including volatile and non-volatile memory and/or storage elements), a graphics adapter, an input interface, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage medium or device (e.g., ROM or magnetic diskette) readable by a general or special purpose programmable computer, for configuring and operating the computer when the storage medium or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

### EXAMPLES

### Example 1: Machine Learning Model Improves Detection of Functional Enhancer Promoter Pairs

### Overview

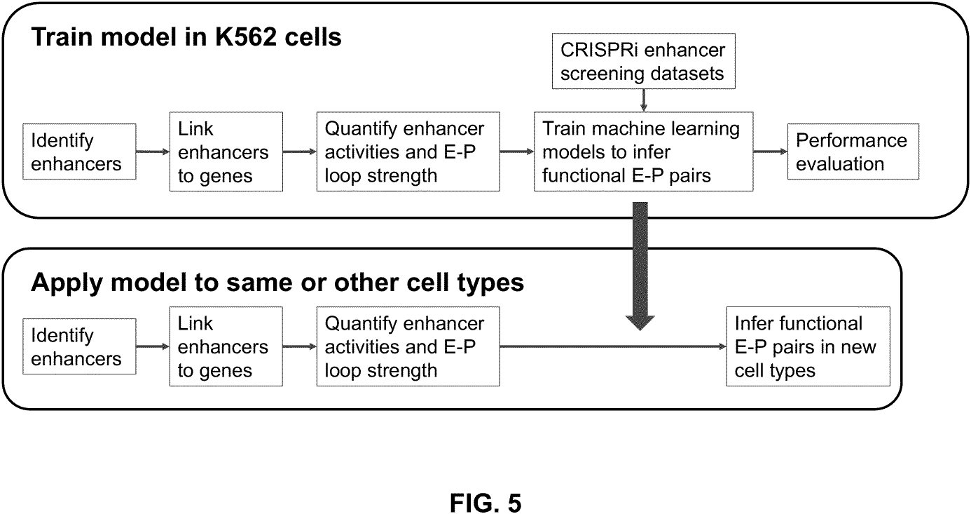

Enhancer-promoter interaction characterization (EPIC) is a machine learning model for predicting functional enhancer-promoter (E-P) pairs. EPIC integrates epigenomic data such as HiC/HiChIP, ChIP-seq, and ATAC-seq, and uses CRISPRi-based enhancer perturbation screening data to train a random forest model to classify E-P pairs as functional or non-functional. FIG. 5 is a diagram showing example training and deployment of a machine learning model for inferring functional enhancer promoter pairs. The models were trained using training data generated from K562 cells and were applied to predict E-P interactions in other cell types.

As shown in FIG. 5, during training of the model, enhancers were first identified. These enhancers were then linked to genes. Values of various features, such as epigenomic features were calculated. For example, feature values include quantified enhancer activities and E-P loop strength. Machine learning models, specifically random forest models, were trained using the feature values as input, as well as CRISPRi enhancer screening datasets, which served as reference ground truths. The trained machine learning models were evaluated for their performance.

During deployment, the trained machine learning models were applied to the same (e.g., K562 cells) or other cell types to predict functional or non-functional enhancer-promoter pairs. Here, enhancers were identified and linked to genes. Feature values including quantified enhancer activities and E-P loop strength were determined. These feature values served as input to the machine learning model to infer whether the enhancer-promoter pair is functional or non-functional.

### Enhancers, Promoters, E-P Interactions

Enhancer candidates were defined in K562 cells as the union of EP300 ChIP-seq peaks and the peaks of ATAC-seq data that overlap with H3K27ac or H3K4me1 ChIP-seq peaks. To improve positional accuracy, the center positions of enhancers and promoters were defined according to single base-pair resolution. The centers of the enhancers were defined as the summit positions (1bp) of MACS peak calls of EP300 ChIP-seq or ATAC-seq. Similarly, the centers of promoter regions represent the transcription start site (1bp) of protein-coding and lncRNA genes (GENCODE v24). The enhancer regions are connected to the promoter regions within 1 Mb genomic distance to define the genome-wide K562 E-P pairs.

### CRISPRi Data

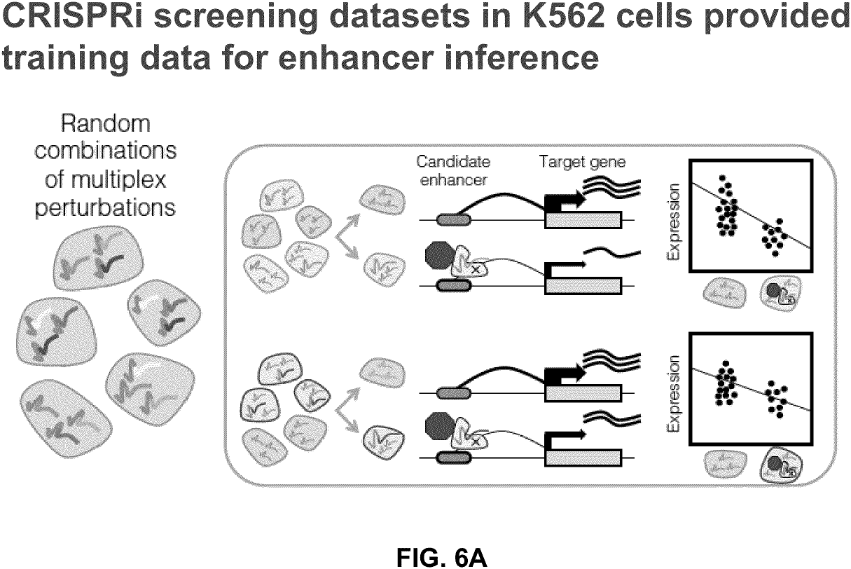

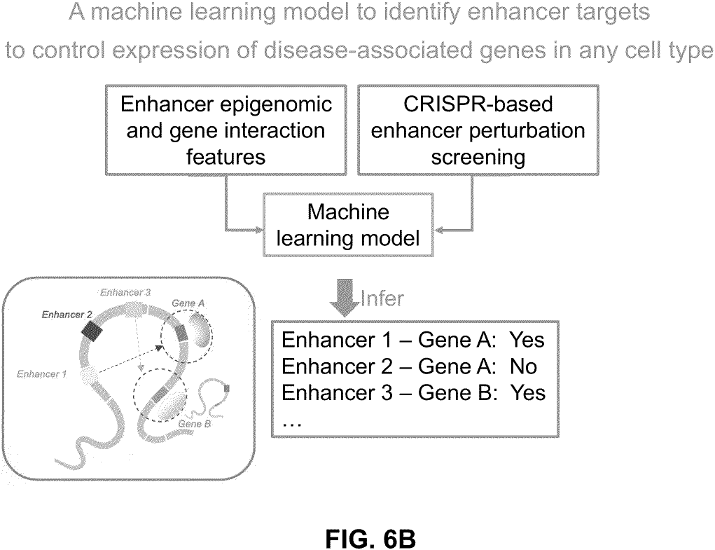

CRISPRi-based enhancer perturbation screening data in K562 cells were curated from the published studies of Xie et al. (2019) Cell Reports 29: 2570-78, and Gasperini et al. (2019). Cell 176: 377-390, each of which is incorporated by reference in its entirety. The enhancer-gene pairs processed by SCEPTRE (Barry et al. (2021) Genome Biol. 22: 344) were used to label the overlapped genome-wide K562 E-P pairs as positive (significant CRISPRi enhancer-gene pairs) and negative (insignificant CRISPRi enhancer-gene pairs). FIG. 6A is an example diagram showing CRISPRi screening for generating training datasets. Here, random combinations of perturbations were imparted to enhancers of cells (e.g., using CRISPRi) to determine whether the perturbations resulted in modulation of target gene expression.

FIG. 6B shows example generation and implementation of a machine learning model to predict enhancer-promoter and enhancer-gene interactions. As described above, the machine learning model was trained using values of features. CRISPRi-based screens identified genes whose expression were regulated or not regulated by certain enhancers, thereby serving as the reference ground truth for training the machine learning model. The trained machine learning model was then deployed to infer functional enhancer-promoter pairs (or as shown in FIG. 6B, functional enhancer-gene pairs, where the gene is under control of a promoter). As shown in FIG. 6B, a functional enhancer-gene pair is identified as “Yes” (such as the pair of Enhancer 1 - Gene A, as well as the pair of Enhancer 3 - Gene B).

### Epigenomic Data Quantification

HiC/HiChIP data were processed using HiC-pro pipeline, which is described in Servant et al. (2015) Genome Biol. 16: 259, the entirety of which is hereby incorporated by reference, and the de-duplicated read-pairs were used to quantify the raw paired-end tag (PET) counts of E-P pairs. The two anchor centers (enhancer and promoter) of each E-P pair were expanded to 5 kb, 10 kb, 15 kb, and 20 kb to count the number of PETs whose two paired-ends overlap with the two anchors of the E-P pairs, respectively. The raw PET counts were normalized by the total number of intra-chromosome PETs and the number of restriction enzyme cut sites within the two anchor regions. The normalized PET counts from the four anchor sizes were used as four features representing chromatin interaction frequencies.

ATAC-seq and ChIP-seq of H3K27ac, H3K4me1, H3K4me3, EP300, CTCF, and input data were aligned using Bowtie2. For paired-end data, reads with the same coordinates in both ends were de-duplicated. For each dataset, the number of reads overlapping the enhancer and promoter regions (expanded to 300bp, 500bp, 1 kb, 2 kb, and 4 kb window size from the center positions) were counted, and the raw read counts were normalized by the total read count of the dataset to generate 10 features representing the biochemical activities of enhancers and promoters. A distance feature was generated by quantifying the genomic distance between the two anchors of the E-P pairs.

### Feature Engineering

To comprehensively characterize the E-P pairs, new features were generated that represent the interaction among the individual features. These include features that quantify genomic distances and chromatin interaction frequencies of E-P pairs, biochemical activities of enhancers and gene promoters, and interactions among these features.

First, an APMI feature was defined as:

$$A P M I = {\left(A T A C ∗ E P 300 ∗ H 3 K 4 m e 1\right)}^{\frac{1}{3}} ∗ H i C h I P$$

where ATAC and EP300 are the normalized read counts in the 1 kb enhancer regions, H3K4me1 is the normalized read counts in 2 kb enhancer regions, and HiChIP is the normalized PET counts in 10 kb anchors.

Based on the APMI feature, a new set of features were generated for quantifying the relative contribution of an enhancer e to a gene g from the gene perspective or enhancer perspective. Specifically, the relative contribution of a particular enhancer e to a gene g from the gene perspective is represented as:

$$f r a c G e n {e}_{e g} = \frac{A P M {I}_{e g}}{{\sum }_{j} A P M {I}_{j g}}$$

where j indexes all the enhancers connected to gene g.

Additionally, the relative contribution of a particular enhancer e to a gene g from the enhancer perspective is represented as:

$$f r a c E n {h}_{e g} = \frac{A P M {I}_{e g}}{{\sum }_{k} A P M {I}_{e k}}$$

where k indexes all the genes connected to enhancer e.

Furthermore, the relative contributions of enhancers to genes from the gene perspective or the enhancer perspective were further combined to form new features including:

$$f r a c G m {E}_{e g} = f r a c G e n {e}_{e g} ∗ f r a c E n {h}_{e g}$$

$$f r a c G p {E}_{e g} = f r a c G e n {e}_{e g} + f r a c E n {h}_{e g}$$

$$a p m i G e n {e}_{e g} = f r a c G e n {e}_{e g} ∗ A P M {I}_{e g}$$

$$a p m i E n {h}_{e g} = f r a c E n {h}_{e g} ∗ A P M {I}_{e g}$$

$$a p m i G m {E}_{e g} = f r a c G m {E}_{e g} ∗ A P M {I}_{e g}$$

$$a p m i G p {E}_{e g} = f r a c G p {E}_{e g} ∗ A P M {I}_{e g}$$

These features were used to train a machine learning model, as described below.

### Model Training and Feature Selection

Random forest models were constructed to classify E-P pairs based on the features described above. The random forest models were trained using five-fold cross-validation using the following data splitting strategy to ensure the independence between samples in training and test set.

The labeled E-P pairs were divided into independent units such that there is not crosstalk between the units, i.e., all the genes and enhancers cannot be present in more than one unit. The units of E-P pairs were then grouped into five groups such that number of E-P pairs, the positive/negative ratios, and the distance distributions are similar across all groups. This data splitting strategy is analogous to the “chromosome-split” strategy (Cao and Fullwood, 2019), but it is not constrained by the large granularity of chromosomes, and leads to independent groups that are more similar to each other.

Next, a strategy for feature selection was implemented to determine the final features in the model. Initially, a random forest model was trained using all the features and the features were sorted based on the feature importance values computed based on the trained model. The top 40 important features were used as the population of a genetic algorithm to select the final features that optimize for both the area under precision-recall (AUPR) and logistic loss metrics.

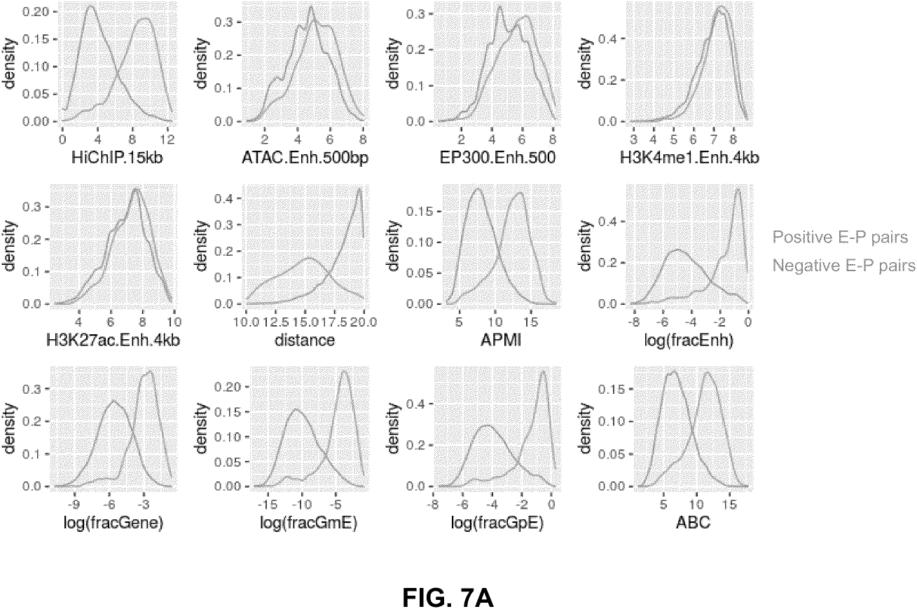

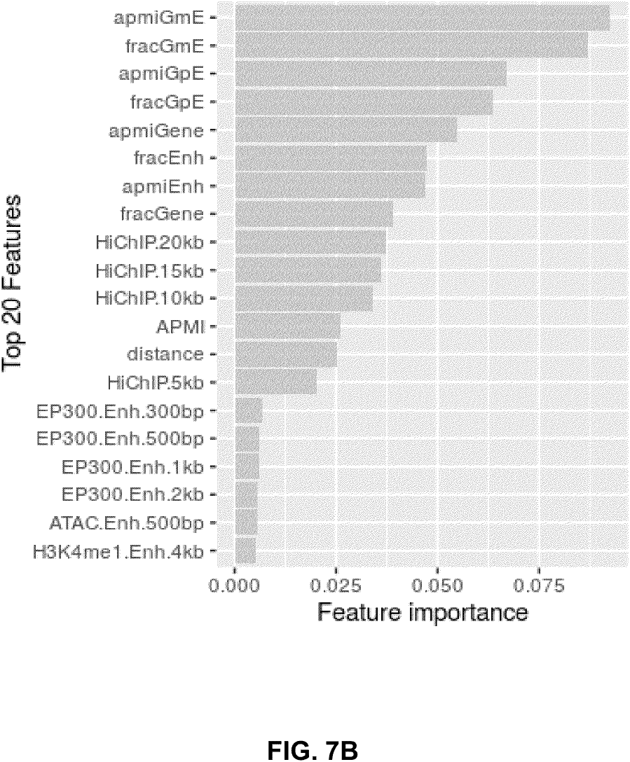

FIG. 7A shows differential features that distinguish functional and non-functional enhancer promoter pairs. Of note, the most informative features that distinguish between functional E-P pairs (identified as Positive E-P pairs in FIG. 7A) and non-functional E-P pairs (identified as Negative E-P pairs in FIG. 7A) include the engineered features representing combination of other features.

FIG. 7B shows ranking of features according to feature importance. Here, the engineered features including apmiGmE, fracGmE, apmiGpE, fracGpE, apmiGene, fracEnh, and fracGene are the highest ranked features, demonstrating the importance of incorporating engineered features.

### Performance of EPIC Model

Two different versions of the EPIC model were constructed and evaluated. A first model, referred to as “EPIC basic” includes 75 features (e.g., features of a first set of features), but does not incorporate additionally engineered features. Specifically, the “EPIC basic” model incorporates the features of:
HiChIP.anchorSize: anchorSize = 5 kb, 10 kb, 15 kb, or 20 kb (n=4)
Assay.Position.windowSize, where Assay=ATAC, H3K27ac, H3K4me1, H3K4me3, EP300, CTCF, or Input; Position = Enhancer or TSS; windowSize = 300bp, 500bp, 1 kb, 2 kb, or 4 kb. (n=7*2*5=70)
Genomic distance (n=1)

A second model, referred to in this example as “EPIC full” incorporates features including the engineered features. Specifically, the “EPIC full” model incorporates the features of:
HiChIP.anchorSize: anchorSize = 5 kb, 10 kb, 15 kb, or 20 kb (n=4)
Assay.Position.windowSize, where Assay=ATAC, H3K27ac, H3K4me1, H3K4me3, EP300, CTCF, or Input; Position = Enhancer or TSS; windowSize = 300bp, 500bp, 1 kb, 2 kb, or 4 kb. (n=7*2*5=70)
Genomic distance (n=1)
and further includes the engineered features of:
APMI
fracEnh
fracGene
fracGmE
fracGpE
apmiGene
apmiEnh
apmiGmE
apmiGpE.

The “EPIC full” and “EPIC basic” models were evaluated for their ability to correctly characterize E-P pairs as functional or non-functional. In particular, the performance of the “EPIC full” and “EPIC basic” models were evaluated in comparison to the ABC model, one of the best performing methods, for predicting functional E-P interactions. The ABC model is further described in Fulco et al. (2019) Nat. Genet. 51: 1664-9, which is incorporated by reference in its entirety.

As a comparison, an ABC feature was constructed as follows:

$$\text{ABC =} {\left(\text{ATAC} \text{.Enhancer} \text{.500bp * H3K27ac} \text{.Enhancer} \text{.500bp}\right)}^{1 / 2} \text{* HiChIP} \text{.5kb}$$

Altogether, the conventional ABC model only uses three features shown in the equation above. In contrast, the EPIC basic model incorporates additional features, while the EPIC full model further incorporates engineered features.

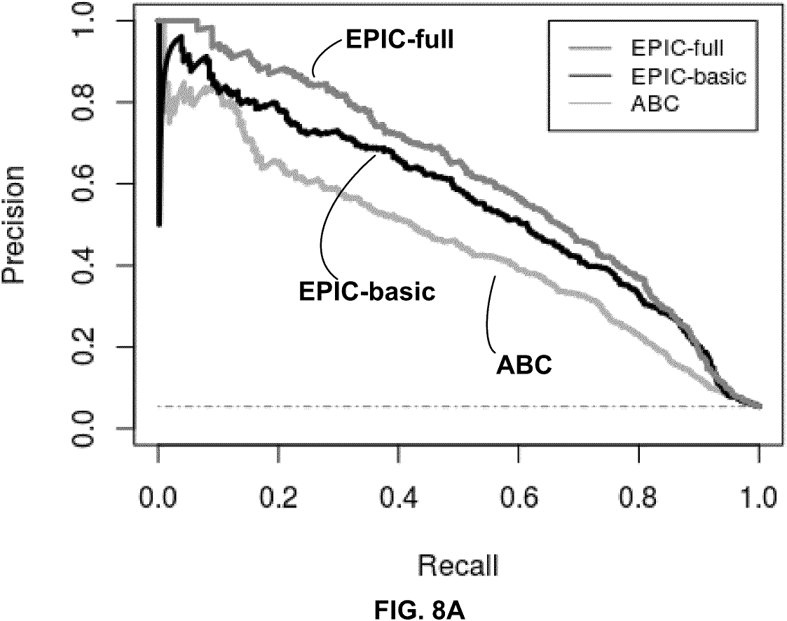

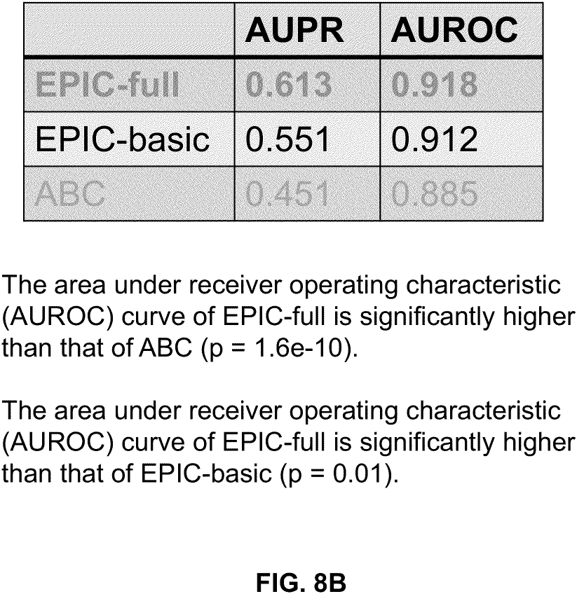

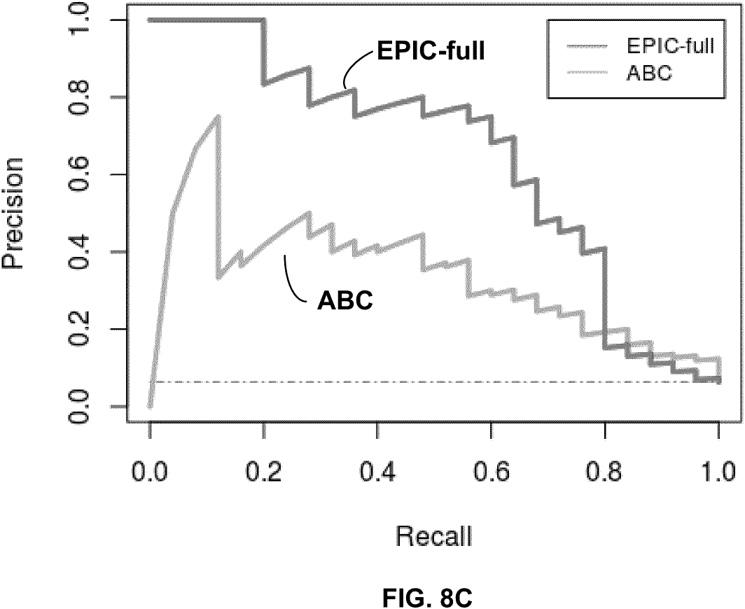

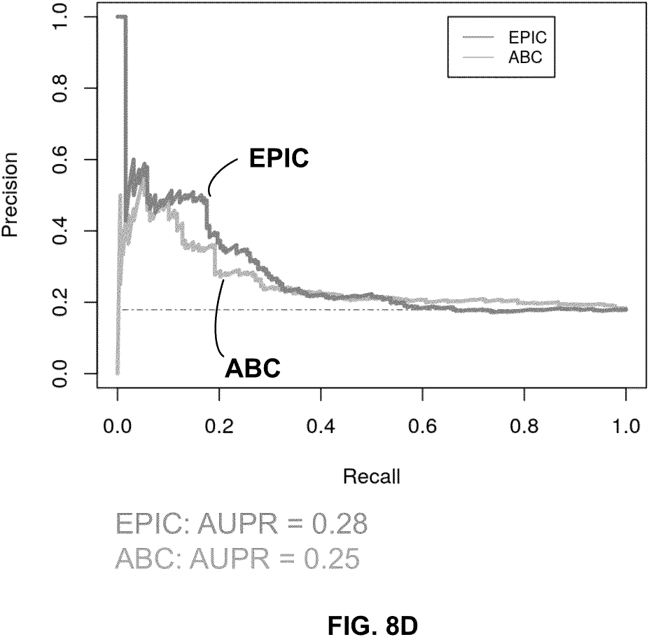

FIG. 8A is a precision-recall curve of the “EPIC full” and “EPIC basic” models in comparison to a state of the art “ABC” model. Additionally, FIG. 8B shows performance of the “EPIC full” and “EPIC basic” models in comparison to a state of the art “ABC” model. Generally, the EPIC full model which incorporates the engineered features achieves improved performance in comparison to both the EPIC basic and the ABC model. Specifically, the EPIC full model achieved an area under precision-recall curve (AUPR) value of 0.613 and area under receiver operating curve (AUROC) value of 0.918. Comparatively, the EPIC basic model achieved an AUPR value of 0.551 and AUROC value of 0.912. This demonstrates that the incorporation of the engineered features by the EPIC full model improves the performance of the model. Furthermore, the ABC model achieved an AUPR value of 0.451 and an AUROC value of 0.885. This demonstrates that the EPIC models (both the EPIC full and EPIC basic models) outperform the state-of-the-art model for predicting functional and non-functional E-P pairs.

Furthermore, to assess the functional relevance of the E-P interactome mapped by EPIC in a new cell type, epigenomic data were generated in human primary hepatocytes. EPIC discovered about 30,000 E-P interactions. Furthermore, in comparing EPIC with the ABC model in discovering target genes of a set of “gold-standard” curated GWAS loci of liver-related diseases and traits, details of which are described in Mountjoy et al. (2021) Nat. Genet. 53: 1527-33, which is incorporated by reference in its entirety, the EPIC full model (80% precision at 50% recall) was more accurate than the ABC model (40% precision at 50% recall) in distinguishing causal genes from neighboring genes. Specifically, FIG. 8C shows performance of EPIC full model in comparison to the ABC model in linking GWAS loci to causal genes. Here, the EPIC full model achieved a AUPRC = 0.643 and AUROC = 0.879, which represents an improvement in comparison to the ABC model which achieved a AUPRC = 0.337 and AUROC = 0.877. Altogether, the EPIC scores significantly associate with the liver eQTL status of the E-P pairs. These results demonstrate the functional relevance of the E-P pairs discovered by the EPIC full model.

In summary, the EPIC full model enables accurate cell-type-specific prediction of functional E-P interactions using epigenomic data. It outperforms an established method in predicting E-P interactions and in linking GWAS loci to causal genes in a new cell type. Applying EPIC to human cell types may help discover disease-causing genes and enable development of novel therapeutics that target enhancers of disease-related genes.

### Performance of Additional Model on HepG2 Cells

To further validate the predictions of trained machine learning models, a new EPIC model was trained and implemented in a new cell type in comparison to the gold-standard ABC model. In particular, the new EPIC model was compared to the ABC model using a set of functional enhancer-promoter pairs discovered in a CRISPRi-based enhancer perturbation screen in HepG2 cells.

Here, the EPIC model refers to the “EPIC full” model which includes the following 75 features (e.g., features of a first set of features):
HiChIP.anchorSize: anchorSize = 5 kb, 10 kb, 15 kb, or 20 kb (n=4)
Assay.Position.windowSize, where Assay=ATAC, H3K27ac, H3K4me1, H3K4me3, EP300, CTCF, or Input; Position = Enhancer or TSS; windowSize = 300bp, 500bp, 1 kb, 2 kb, or 4 kb. (n=7*2*5=70)
Genomic distance (n=1)
The “EPIC full” model further includes the engineered features of:
APMI
fracEnh
fracGene
fracGmE
fracGpE
apmiGene
apmiEnh
apmiGmE
apmiGpE.

FIG. 8D shows the performance of the EPIC model in comparison to the gold-standard ABC model. Here, the EPIC model achieves an AUPR value of 0.28, thereby outperforming the ABC model which achieved an AUPR value of 0.25. The results here indicate that the EPIC model can accurately predict functional E-P pairs across multiple cell types.

### Example 2: Linking Variants and Enhancer Promoter Pairs With Putative Target Genes

Functional E-P pairs were identified in liver cells using the methods described in Example 1. Furthermore, the E-P pairs were analyzed in relation to lead and fine-mapped single nucleotide polymorphisms representing associations from liver-related genome wide association studies (GWAS) studies.

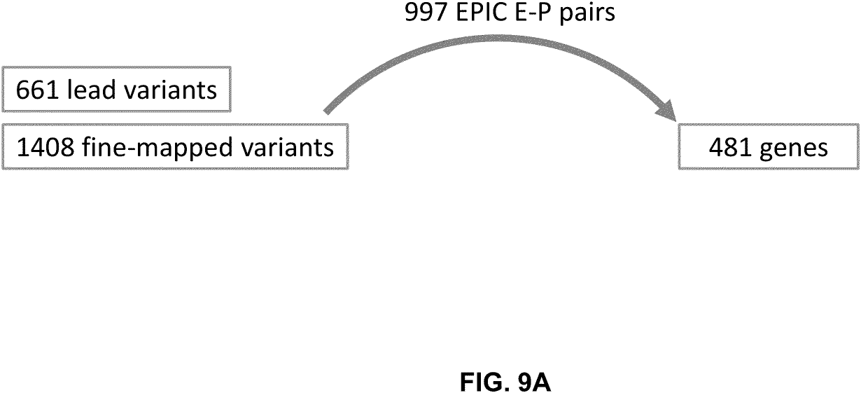

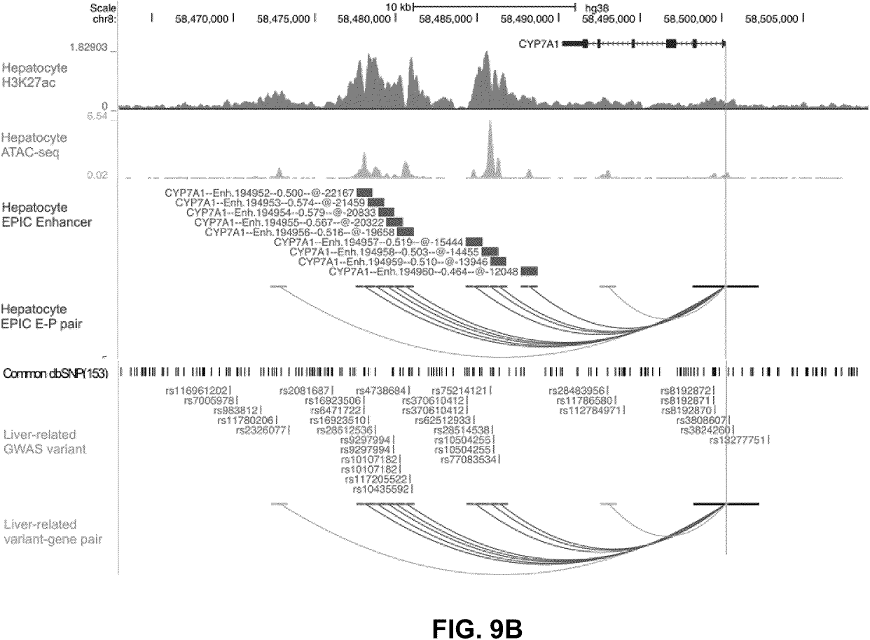

Specifically, FIG. 9A shows the overlapping of E-P pairs and liver-related GWAS loci associations to putative target genes. Here, 997 E-P pairs were identified according to the methods of Example 1. Furthermore, a total of 1408 fine-mapped variants from the GWAS study were obtained. The overlapping putative target genes of the E-P pairs and fine-mapped variants revealed a total of 481 genes.

FIG. 9B further depicts the separate analysis of E-P pairs and GWAS variants and their respective associations from 32 genome wide association studies (GWAS) studies of cholesterol (total, LDL, HDL), triglyceride levels, cholelithiasis, and cholestasis, with a particular putative target gene, CYP7A1. CYP7A1 is a well characterized enzyme regulator of bile acid and cholesterol homeostasis and these enhancers have been experimentally validated. Here, the first row identifies active H3K27ac enhancer marks in hepatocytes. The second row identifies peaks in chromatin accessibility data (ATAC-seq data). Together, the H3K27ac peaks and ATAC-seq peaks were analyzed using machine learning model disclosed herein to identify presence of hepatocyte enhancers, shown in the third row in FIG. 9B. These identified enhancers were linked to promoters of a target gene to identify hepatocyte E-P pairs, as shown in the fourth row in FIG. 9B. The fifth row of FIG. 9B (entitled “Common dbSNP”) identifies common single nucleotide polymorphisms from the dbSNP database. The sixth row identifies liver-related GWAS variants and the seventh row identifies the mapping of the GWAS variants to a target putative gene. Thus, altogether, FIG. 9B shows the overlapping E-P pairs and variant-gene pairs.

Combining GWAS and E-P pair analysis enables the discovery of disease-causing genes, which will further enable development of novel therapeutics that target enhancers of disease-related genes.

### Example 3: Additional Models Outperform ABC Model

Three additional different versions of the EPIC model were constructed and evaluated in comparison to the ABC model. The three models, referred to as “Model A,” “Model B,” and “Model C,” are shown below in Table 1. The corresponding features (including both basic feature(s) and engineered features) of each of the EPIC models are further documented in Table 1.

TABLE 1

Three versions of the EPIC model incorporating different subsets of basic and engineered features

| EPIC Model | Basic Features | Engineered Features | AUPR |
|---|---|---|---|
| EPIC Model A | 75 basic features | APMI, fracEnh, fracGene | 0.615 |
| EPIC Model B | 35 basic features derived from ATAC, EP300, H3K4me1, HiChIP, and genomic distance | APMI, fracEnh, fracGene | 0.610 |
| EPIC Model C | 1 basic feature including genomic distance | APMI, fracEnh, fracGene | 0.586 |

The first model (EPIC Model A) includes 75 basic features of:
HiChIP.anchorSize: anchorSize = 5 kb, 10 kb, 15 kb, or 20 kb (n=4)
Assay.Position.windowSize, where Assay=ATAC, H3K27ac, H3K4me1, H3K4me3, EP300, CTCF, or Input; Position = Enhancer or TSS; windowSize = 300bp, 500bp, 1 kb, 2 kb, or 4 kb. (n=7*2*5=70)
Genomic distance (n=1)

Furthermore, the first model (EPIC Model A) includes 3 engineered features including: APMI, fracEnh, and fracGene. Thus, in comparison to the EPIC Full model (described in Example 1) which incorporated 8 engineered features, EPIC Model A incorporates only 3 engineered features.

The second model (EPIC Model B) includes 35 basic features of:
HiChIP.anchorSize: anchorSize = 5 kb, 10 kb, 15 kb, or 20 kb (n=4)
Assay.Position.windowSize, where Assay=ATAC, H3K4me1, EP300; Position = Enhancer or TSS; windowSize = 300bp, 500bp, 1 kb, 2 kb, or 4 kb. (n=3*2*5=30)
Genomic distance (n=1)

Furthermore, the second model (EPIC Model B) includes 3 engineered features including: APMI, fracEnh, and fracGene. Thus, in comparison to the EPIC Model A, EPIC Model B incorporates a subset of the basic features.

The third model (EPIC Model C) includes 1 basic feature of:
Genomic distance (n=1)

Furthermore, the third model (EPIC Model C) includes 3 engineered features including: APMI, fracEnh, and fracGene. Thus, in comparison to the EPIC Model A, EPIC Model B incorporates an even further reduced subset of the basic features (only 1 basic feature of genomic distance).

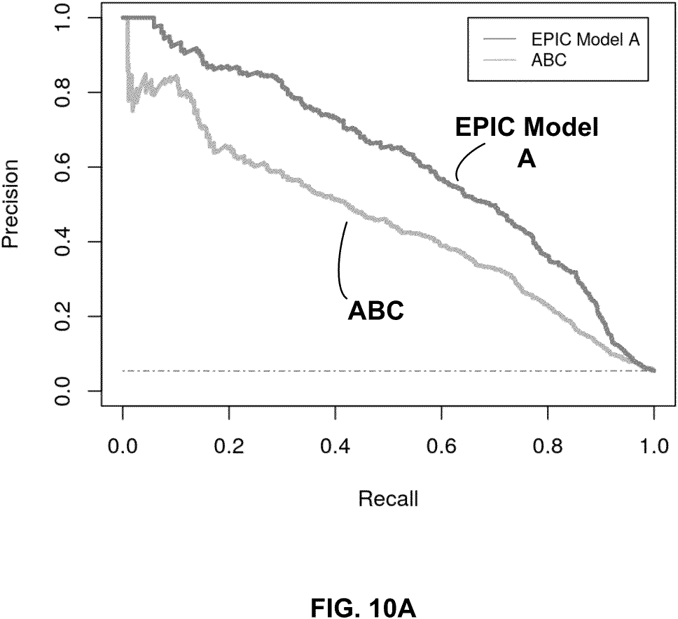

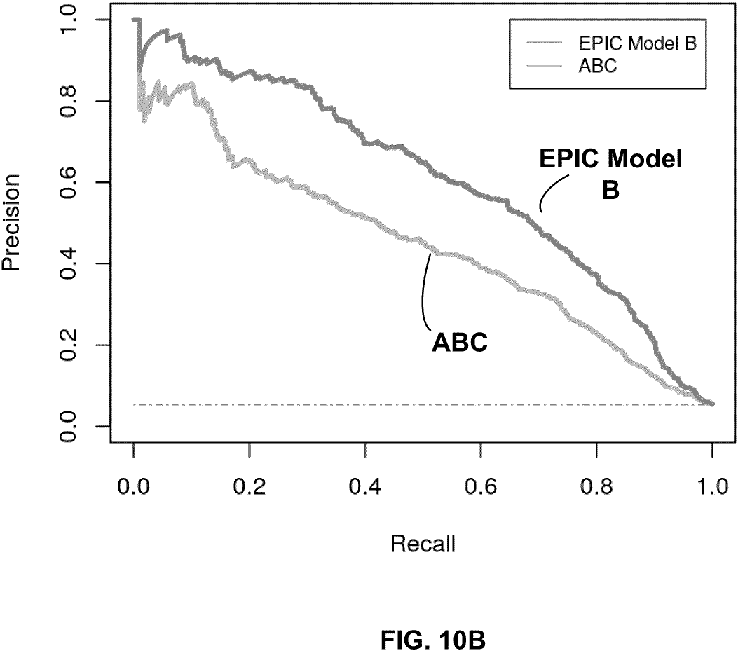

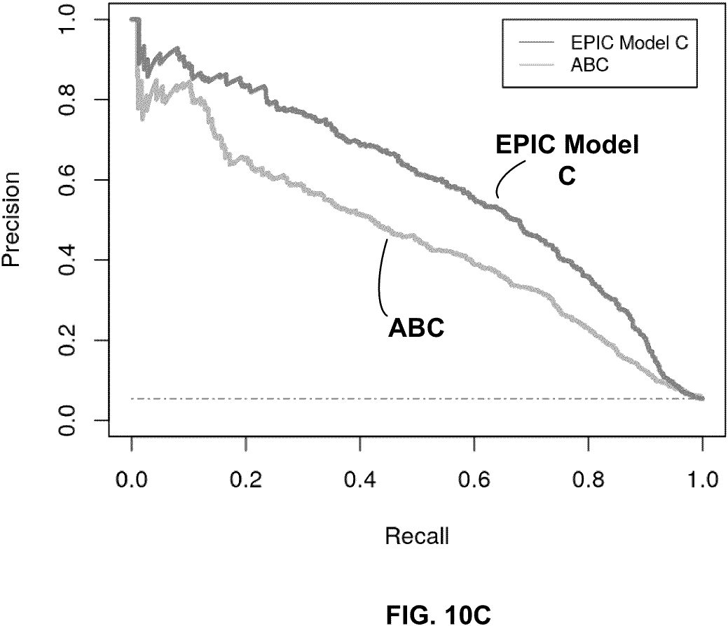

As shown in Table 1 and FIGS. 10A-10C, each of EPIC Model A, EPIC Model B, and EPIC Model C achieved predictive performance. Specifically, EPIC Model A achieved an area under precision recall (AUPR) curve values of 0.615. EPIC Model B achieved an area under precision recall (AUPR) curve values of 0.610. EPIC Model C achieved an area under precision recall (AUPR) curve values of 0.586. Each of these outperformed the ABC model which achieved an AUPR value of 0.451.

Altogether, the results indicate that the various EPIC models that incorporate reduced subsets of basic features (e.g., N=35 basic features in EPIC model B or N=1 basic feature in EPIC Model C) or reduced set of engineered features (e.g., N=3 engineered features of APMI, fracEnh, and fracGene in each of EPIC model A, EPIC model B, and EPIC model C) exhibit improved performance in comparison to the gold standard ABC model.

1. A method, comprising:
obtaining a dataset comprising epigenomic data for one or more enhancer-promoter pairs;
for the one or more enhancer-promoter pairs:
generating, from the dataset comprising epigenomic data, values for a plurality of features comprising a first set of features and a second set of features of the enhancer-promoter pair by:
generating values for the first set of features; and
generating values for the second set of features engineered from subsets of the first set of features;
applying a machine learning model to analyze the values for the plurality of features of the one or more enhancer-promoter pairs; and
determining whether one of the one or more enhancer-promoter pairs is a functional enhancer-promoter pair based on an output of the machine learning model.

2. The method of claim 1, wherein the second set of features engineered from subsets of the first set of features comprise an enhancer contribution feature that quantifies relative contribution of the enhancer across a plurality of enhancers to a gene operably controlled by the promoter.

3. The method of claim 2, wherein the second set of features further comprise a composite feature of the enhancer representing a combination of an ATAC feature, an EP300 feature, a H3K4me1 feature, and a HiChIP feature.

4. The method of claim 3, wherein the enhancer contribution feature is a ratio of the composite feature of the enhancer to a combination of a plurality of composite features for the enhancer.

5. The method of claim 1, wherein the second set of features engineered from subsets of the first set of features comprise a gene contribution feature that quantifies relative contribution to a gene operably controlled by the promoter across a plurality of genes influenced by the enhancer.

6. The method of claim 5, wherein the second set of features further comprise a composite feature of the gene representing a combination of an ATAC feature, an EP300 feature, a H3K4me1 feature, and a HiChIP feature.

7. The method of claim 6, wherein the gene contribution feature is a ratio of the composite feature of the gene to a combination of a plurality of composite features for the gene.

8. (canceled)

9. The method of claim 1, wherein the second set of features comprise APMI, fracEnh, and fracGene features.

10. (canceled)

11. The method of claim 1, wherein the second set of features comprise APMI, fracEnh, fracGene, fracGmE, fracGpE, apmiGene, apmiEnh, apmiGmE, and apmiGpE features.

12. (canceled)

13. The method of claim 12, wherein the first set of features comprise features of ATAC, EP300, H3K4me1, HiChIP, and genomic distance.

14. (canceled)

15. The method of claim 1, wherein at least one feature of the second set has a higher feature importance value in comparison to at least one feature of the first set.

16-22. (canceled)

23. The method of claim 1, wherein the machine learning model is a random forest model.

24. The method of claim 1, wherein the dataset comprises one or more of:
chromatin accessibility data identifying chromatin-accessible regions across the genome; and
chromatin binding data identifying chromatin interactions.

25. The method of claim 24, wherein the chromatin accessibility data comprises DNase-seq or ATAC-seq data.

26. The method of claim 24, wherein the chromatin binding data comprises data for one or more of:
DNA-DNA interactions;
chromatin domains;
protein-chromatin binding sites; and
transcription factor binding motifs.

27. The method of claim 24, wherein the chromatin binding data comprising HiChIP or ChIP-seq data.

28. The method of claim 24, wherein the chromatin binding data comprises data for one or more active enhancer marks.

29. The method of claim 28, wherein the one or more active enhancer marks comprise EP300, H3K27ac, or H3K4me1.

30. The method of claim 24, wherein the chromatin binding data comprises data for one or more repressive factors.

31. The method of claim 30, wherein the one or more repressive factors comprise H3K27me3, H3K9me3, H4K20me1, NCOR1, HDAC1/2/3, EZH2, SUZ12, ZEB2, or REST.

32-70. (canceled)
